(19) United States
(12) Patent Application Publication
(10) Pub. No.: US 2024/0009137 A1
(43) Pub. Date: Jan. 11, 2024

(54) ORAL THERAPEUTIC DELIVERY

(71) Applicant: ROYAL MELBOURNE INSTITUTE OF TECHNOLOGY, Melbourne, Victoria (AU)

(72) Inventors: Charlotte Elizabeth CONN, Melbourne, Victoria (AU); Jamie STRACHAN, Melbourne, Victoria (AU)

(21) Appl. No.: 18/254,983

(22) PCT Filed: Nov. 30, 2021
(86) PCT No.: PCT/AU2021/051428

§ 371 Date: May 30, 2023

(30) Foreign Application Priority Data
Nov. 30, 2020 (AU) 2020904426
Feb. 12, 2021 (AU) 2021900348

(51) Int. Cl. A61K 9/50; A61K 38/28

(52) U.S. Cl. CPC A61K 9/5084; A61K 38/28; A61K 9/5015

(57) ABSTRACT

A dosage form for oral delivery of a therapeutic agent to a subject, the dosage form comprising: (i) a lipid nanocarrier formulation, the lipid nanocarrier formulation comprising the therapeutic agent, and lipids in the form of a mesophase; and (ii) an enteric coating encapsulating the lipid nanocarrier formulation.

This application claims priority from Australian provisional patent application no. 2020904426 filed on 30 Nov. 2020 and Australian provisional patent application no. 2021900348 filed on 12 Feb. 2021, the contents of which are to be taken as incorporated herein by this reference.

### TECHNICAL FIELD

The present disclosure relates to formulations for oral delivery of a therapeutic agent, methods of preparing the formulations and uses thereof.

### BACKGROUND OF INVENTION

Intravenous (IV), intramuscular (IM), and subcutaneous (SC) are the three most frequently used injection routes in drug delivery.

The absorption mechanism as well as the nature of the drug are the fundamental factors that determine the appropriate delivery systems for achieving the highest bioavailability and effectivity. For example, depending on the circumstances, insulin can be injected 1) subcutaneously (in the skin) via an insulin syringe, pre-filled pen device or insulin pen; 2) for certain patients with type 1 diabetes, it can be delivered as an insulin infusion via a wearable personal insulin pump; or 3) be administered through an intravenous insulin infusion.

For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm. Intravenous administration is the best way to deliver a precise dose quickly and in a well-controlled manner throughout the body.

When given intravenously, a drug is delivered immediately to the bloodstream and tends to take effect more quickly than when given by any other route.

For the subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. Protein drugs that are large in size, such as insulin, usually reach the bloodstream through the lymphatic vessels because these drugs move slowly from the tissues into capillaries. The subcutaneous route is used for many protein drugs because such drugs would be destroyed in the digestive tract if they were taken orally.

The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed.

Drugs administered by IV, SC or IM avoid the gastrointestinal (GI) environment. However, the injection can cause significant problems, including needle-associated phobia and pain, unsafe needle use and improper disposal, the need for trained healthcare personnel, muscle atrophy, and injuries to bones and nerves.

Because of these problems, oral drug delivery remains the preferable route of drug administration. However, not all drugs possess the desirable physicochemical and pharmacokinetic properties which favour oral administration mainly due to poor bioavailability. This has in some cases led to the choice of other routes of administration, which may compromise patient convenience and increase the risk of non-compliance. Poor bioavailability has necessitated the administration of higher than normally required oral doses which often leads to economic wastages, risk of toxicity, erratic and unpredictable responses. There remains a need to provide drug delivery of dosage forms that enhances the oral bioavailability of drugs.

### SUMMARY OF INVENTION

Surprisingly and as disclosed herein an oral delivery dosage form that increases the bioavailability of poorly absorbed therapeutic agents has been developed; in order to increase their clinical efficacy when administered orally. The dosage form comprises formulation of a therapeutic agent in a lipid nanocarrier. This formulation is then encapsulated in an enteric coating. It has been surprisingly demonstrated that this dosage form can be used to improve the absorption and bioavailability of insulin after oral administration.

It has further surprisingly found that this dosage form can also be used to improve the absorption and bioavailability of therapeutic agents such as proteins, for example hormones, and small molecules, for example antibiotics, after oral administration.

Accordingly, in one aspect the present disclosure relates to dosage form for oral delivery of a therapeutic agent to a subject, the dosage form comprising: a lipid nanocarrier formulation, the lipid nanocarrier formulation comprising the therapeutic agent; and an enteric coating encapsulating the lipid nanocarrier formulation.

A further aspect of the present disclosure relates to a dosage form for oral delivery of a therapeutic agent to a subject, the dosage form comprising a lipid nanocarrier formulation, the lipid nanocarrier formulation comprising the therapeutic agent, and lipids in the form of a mesophase; and an enteric coating encapsulating the lipid nanocarrier formulation.

In some embodiments, the lipid nanocarrier formulation comprises lipids in the form of a mesophase.

The lipid mesophase may comprise, for example, the reverse bicontinuous cubic phase or the bicontinuous cubic phase.

In other embodiments, the mesophase may comprise the reverse hexagonal phase.

In other embodiments the mesophase may comprise a reverse bicontinuous cubic phase, a primitive cubic phase, double diamond cubic phase, a gyroid cubic phase, an hexagonal phase, a reverse hexagonal phase, cubosomes or hexosomes.

In some embodiments, the mesophase may comprise cubosomes or hexosomes. Therefore, other embodiments relate to the mesophase being structured as a cubosome or a hexosome.

In some embodiments, the lipid nanocarrier comprises a lipid selected from the group consisting of a mono-, di-, or tri-substituted glycerol, charged lipid, branched lipid and a glycolipid. In one embodiment, the lipid nanocarrier is a long chain lipid.

In some embodiments the charged lipid is dioleoyl-3-trimethylammonium propane (DOTAP) present in an amount of up to and including 10% of the lipid nanocarrier formulation.

Such lipids may advantageously direct transfer of the insulin or derivatives thereof into the lymph system avoiding the first-pass metabolism and improving bioavailability.

In other embodiments, the lipid nanocarrier comprises lipids of the following formula I:

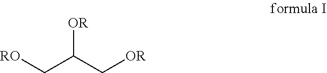

wherein at least one R is formula II, and the reaming R groups are independently selected from a hydrogen or formula II:

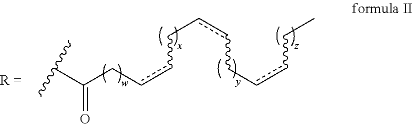

wherein w, x, y and z are independently selected from the group consisting of 0, 1, 2, 3, 4, 5, 6, 7, 8, 9, 10, 11 and 12; wherein a broken line represents the presence or absence of a bond; and wherein a wavy bond indicates E or Z bond geometry in the presence of a bond.

In other embodiments, the lipid nanocarrier comprises lipids of the following formula III:

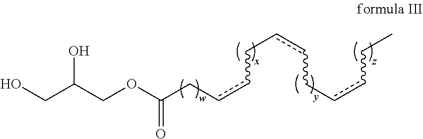

wherein w, x, y and z are independently selected from the group consisting of 0, 1, 2, 3, 4, 5, 6, 7, 8, 9, 10, 11 and 12; wherein a broken line represents the presence or absence of a bond; and wherein a wavy bond indicates E or Z bond geometry in the presence of a bond.

In some embodiments, the lipid nanocarrier comprises lipids selected from the following group: monoolein, phytantriol and monopalmitolein.

In some embodiments, monoolein is present in about 40% to 80% weight of the formulation.

In some embodiments, phytantriol is present in about 60% to 75% weight of the formulation.

In some embodiments, the therapeutic agent is selected from the list consisting of insulin or derivative thereof, a steroidal hormone, antimicrobial such as an antibiotic, a protein such as a hormone, and a peptide such as a neuropeptide.

In some embodiments, the insulin or derivative thereof is selected from the group consisting of glargine (Lantus, Basaglar, Toujeo), detemir (Levemir), degludec (Tresiba), NPH (Humulin N, Novolin N, Novolin ReliOn Insulin N), rapid acting insulin and short acting insulin.

In some embodiments, the insulin or derivative thereof is present in the formulation from 0.01% weight to 1% weight of the formulation.

In some embodiments, the nanocarrier comprises aqueous channels of sizes 1 nm to 17 nm. The therapeutic agent, for example, insulin, may be entrapped within the aqueous channels of the nanocarrier.

In some embodiments, the enteric coating is soluble at a range of about pH 4.5 to pH 7.2.

In other embodiments, the enteric coating is soluble at range of about pH 5.0 to pH 6.0 Advantageously, the solubility of the enteric coating at these pH ranges may allow for dissolution of the coating in the small intestine.

In other embodiments, the enteric coating has a thickness in a range of 0.01 nm to 1.00 mm.

In some embodiments the lipid nanocarrier formulation is encapsulated in the enteric coating, the enteric coating has a thickness in a range of 0.07 mm-0.4mm.

In some embodiments the lipid nanocarrier formulation is aqueous. In further embodiments the lipid nanocarrier formulation has a water content in a range of 1% to 70% weight of the lipid nanocarrier formulation.

In some embodiments the lipid nanocarrier formulation has a water content in a range of up to and including 48% weight of the lipid nanocarrier formulation and wherein the lipid is monoolein, or has a water content in a range of up to and including 48% weight of the lipid nanocarrier formulation and wherein the lipid is phytantriol.

Advantageously, such aqueous lipid nanocarrier formulation may aid in ameliorating degradation of the therapeutic agent in vivo such as in the human gastrointestinal tract.

In other embodiments, the dosage form may be a capsule comprising a filling comprising the lipid nanocarrier formulation and a shell encapsulating the filling, the shell comprising the enteric coating.

In other embodiments, the shell is coated with an enteric coating on at least one of a shell surface facing the filling and an outer shell surface.

In other embodiments the enteric coating on the shell surface facing the filling and on the outer shell surface each independently have a thickness in a range of 30 μm to 380 μm.

In further embodiments, the lipid nanocarrier formulation further comprises a swelling agent. In some embodiments, the formulation has a water content in a range of up to and including the 70% weight of the lipid nanocarrier formulation.

Another aspect of the present disclosure relates to a method for preparing the dosage form comprising the steps of providing the lipid nanocarrier formulation and encapsulating the lipid nanocarrier formulation in an enteric coating.

In some embodiments, the method comprises contacting the lipid, the therapeutic agent and an aqueous solvent under conditions sufficient to, for example, promote self-assembly of the lipid into a mesophase.

In some embodiments, a lipid to aqueous solvent ratio of about 60:40 w/w is used.

In some embodiments, high-pressure homogenisation is used to promote self-assembly of the lipid into a mesophase.

In some embodiments the enteric coating is applied to at least one of the shell surface facing the filling and the outer shell surface. In alternate embodiments the enteric coating may be applied directly to the lipid nanocarrier formulation.

In another aspect, the present disclosure provides a dosage form as described for use in the treatment of diabetes mellitus, wherein the therapeutic agent is insulin or a derivative thereof.

In another aspect, the present disclosure provides a dosage form as described for use treatment or prevention of diabetes mellitus, a condition mediated by bacterial infection, a condition mediated by human growth hormone, or a condition mediated by blood coagulation and wherein the therapeutic agent is present in a therapeutically or prophylactically effective amount.

In another aspect, the present disclosure provides a method for treating or preventing diabetes mellitus which comprises administering to a subject in need, the dosage form as described, wherein the therapeutic agent is insulin or a derivative thereof.

In another aspect, the present disclosure provides method for treating or preventing diabetes mellitus, a condition mediated by bacterial infection, a condition mediated by human growth hormone, or a condition mediated by blood coagulation which comprises administering to a subject in need the dosage form as described and wherein the therapeutic agent is present in a therapeutically or prophylactically effective amount.

In another aspect, the present disclosure provides a use of a therapeutic agent in the manufacture of the dosage form as described for the treatment or prevention of diabetes mellitus, wherein the therapeutic agent is insulin or a derivative thereof.

In another aspect, the present disclosure provides a use of the dosage form as described in the manufacture of a medicament for the treatment or prevention of diabetes mellitus, a condition mediated by bacterial infection, a condition mediated by human growth hormone, or a condition mediated by blood coagulation and wherein the therapeutic agent is present in a therapeutically or prophylactically effective amount.

The present disclosure is not to be limited in scope by the specific examples described herein, which are intended for the purpose of exemplification only. Functionally-equivalent products, compositions and methods are clearly within the scope of the present disclosure.

Any example/embodiment of the present disclosure herein shall be taken to apply mutatis mutandis to any other example/embodiment of the disclosure unless specifically stated otherwise.

### BRIEF DESCRIPTION OF DRAWINGS

FIG. 1. Is a diagram showing examples of where, without wishing to be bound by theory, the lipid nanocarrier promotes lymphatic uptake of therapeutic agent, in the small intestine, for example.

FIG. 2. Is a diagram of a particular set of examples showing a dosage form comprising the formulation of (1) the lipid nanocarrier comprising the therapeutic agent (2); and (3) the enteric coating. The diagram is representative of particular embodiments in which the lipid nanocarrier is in the bicontinuous cubic phase.

FIG. 3. Panel (A) Blood fluorescence of green fluorescent protein (GFP) in blood plasma determined from fluorescence measurements (470/515 Excitation/Emission) over a 6-hour time period following SC injection of 500 μL of GFP (100 μg/ml) of in Sprague Dawley rats. Panel (B) Blood fluorescence of GFP in blood plasma of Sprague Dawley rats determined from fluorescence measurements (470/515 Excitation/Emission) over a 6-hour time period following administration of 50 μg GFP in a lipid cubic phase contained within an enteric capsule Panel (C) Average blood fluorescence of GFP in Sprague Dawley rats over a 6-hour time period following administration via SC injection or enteric capsule/cubic phase. Panel (D) GFP fluorescence (470/515 Excitation/Emission) of blood plasma taken from Sprague Dawley rats taken over a 6-hour time period for all samples.

FIG. 4. The change in blood glucose levels with time for four (4) individual rats (Rats 2, 4 9 and 12 from the subsequent trials) over 24 hours post administration of 15 IU Actrapid insulin by SC injection. Data are shown for two different days for each rat (triangle and circle symbols). The average increase in blood glucose was determined from data between 450 and 1450 mins.

FIG. 5. The change in blood glucose levels with time following delivery of fast acting (Actrapid) insulin via SC injection (circles) and a perforated lipid cubic phase filled enteric capsule (triangles). The estimated blood glucose (BG) increase of Rat 1 over this time period (dashed line) is based on data in FIG. 4.

FIG. 6. The change in blood glucose levels with time following delivery of fast acting (Actrapid) insulin via SC injection (circles) and lipid cubic phase filled enteric capsule (triangles). The estimated blood glucose (BG) increase of Rats 2-4 over this time period (dashed line) is based on data in FIG. 4.

FIG. 7. The change in blood glucose levels with time following delivery of fast acting (Actrapid) insulin via SC injection (circles) and via lipid cubic phase filled enteric capsule (triangles) for four (4) rats (Rats 5-8). The estimated blood glucose (BG) increase of Rats 5-8 over this time period (dashed line) is based on data in FIG. 4.

FIG. 8. The change in blood glucose levels with time following delivery of slow acting (Levemir) insulin via SC injection (circles) and lipid cubic phase filled enteric capsule (triangles) for six (6) rats (Rats 9-14). The estimated blood glucose (BG) increase of Rats 9-14 over this time period (dashed line) is based on data in FIG. 4.

FIG. 9. (A) The average blood glucose level with time (Phase 1) following the delivery of fast-acting (Actrapid) Insulin (1 U) via SC injection (triangles) and via lipid cubic phase filled enteric capsule (hollow circles) (Rats 2-4). Results from Rat 1 (capsule) were not used due to the perforation of the capsule. (B) The average blood glucose level with time (Phase 1) following the delivery of slow acting (Levemir) insulin (1 U) via SC injection (stars) and via lipid cubic phase filled enteric capsule (filled circles) (Rats 9-10). (C) The average blood glucose level with time (Phase 2) following the delivery of fast acting (Actrapid) insulin (1 U) via SC injection (triangles) and via lipid cubic phase filled enteric capsule (hollow circles) (Rats 5-8). (D) The average blood glucose level with time (Phase 2) following the delivery of slow acting (Levemir) insulin (1 U) via SC injection (stars) and via lipid cubic phase filled enteric capsule (filled circles) (Rats 11-14). All blood glucose readings were normalized to an initial BG level of 0.

FIG. 10. Animal trial results showing human growth hormone (HGH) found in blood plasma taken from Sprague Dawley rats over 5 hours. (A) Shows the measured concentration of in blood plasma over a period of 300 min (5 hours) following administration by SC injection. For all four rats the drug concentration in the blood plasma immediately increases to a maximum value in the range 20-32 ng/ml at 60 mins, followed by a gradual decrease to 0 ng/ml over the subsequent 4 hours with baseline drug concentrations reached by 300 min. Figure (B) shows the measured concentration of HGH in blood plasma over a period of 300 min (5 hours) following administration by oral capsule.

FIG. 11. Animal trial results showing human coagulation factor (HCFX) found in blood plasma taken from Sprague Dawley rats over 5 hours. (A) shows the measured concentration of HCFX in blood plasma over a period of 300 min (5 hours) following administration by SC injection. For all four rats, the drug concentration in the blood plasma immediately increases to a maximum value in the range 9-10 ng/ml at 30 mins, followed by a reasonably sharp decrease to 1 ng/ml over the subsequent 90 min. (B) Shows the measured concentration of HCFX in blood plasma over a period of 300 min (5 hours) following administration by oral capsule.

FIG. 12. Animal trial results showing vancomycin found in blood plasma taken from Sprague Dawley rats over 5 hours. (A) shows the measured concentration of vancomycin in blood plasma over a period of 300 min (5 hours) following administration by SC injection. For all four rats, the drug concentration in the blood plasma immediately increases to a maximum value of approximately 1500 ng/ml at 30 mins, followed by a reasonably sharp decrease to baseline values by 180 min. (B) shows the measured concentration of vancomycin in blood plasma over a period of 300 min (5 hours) following administration by oral capsule.

FIG. 13. Animal trial results showing meropenem found in blood plasma taken from Sprague Dawley rats over 5 hours. (A) shows the measured concentration of meropenem in blood plasma over a period of 300 min (5 hours) following administration by SC injection. For all four rats the drug concentration in the blood plasma immediately increases to a maximum value in the range 37-40 ng/ml at 30 mins, followed by a gradual decrease to baseline values by 240 min. (B) shows the measured concentration of vancomycin in blood plasma over a period of 300 min (5 hours) following administration by oral capsule.

FIG. 14. 1D SAXS pattern for human growth hormone (1 mg/ml) encapsulated in MO at an aqueous phase content of 38%.

FIG. 15. 1D SAXS patterns for human coagulation factor X (1 mg/ml)

encapsulated in MO at an aqueous phase content of 38%. The lattice parameter of the QIID phase is calculated to be 103.0 Å.

FIG. 16. 1D SAXS patterns for vancomycin (15 mg/ml) encapsulated in MO at an aqueous phase content of 38%.

FIG. 17. (Left) Double enteric (Eudragit L 100) coated capsule still stable after 24 hours in pH 4 inside and outside. (middle) standard capsule empty showing t=0. (right) single outside coating with pH 4 media inside and outside, showing destruction of the capsule after 8 hours.

FIG. 18. The phase adopted and associated lattice parameter of the MO and PT bulk cubic phase following the addition of insulin at a range of concentrations from 0 to 10 mg/ml at 25° C. The water content of the bulk phase was 48% for MO and 28% for PT. Error bars represent the standard deviation from three (3) repeat experiments.

FIG. 19. 1D SAXS patterns of intensity vs q for MO and PT bulk cubic phase with encapsulated insulin at a range of concentrations between 0 and 10 mg/ml.

FIG. 20. (A) The percentage of encapsulated insulin (2 mg/ml) released from MO with time. Data were fit to the Ritger-Peppas model in (B) (solid line). The percentage of encapsulated insulin (2 mg/ml) released from MO as a function of time. Data were fit to the Higuchi model for release of the first 60% of encapsulated insulin (solid line). (C) The percentage of encapsulated insulin (2.5 mg/ml) released from PT with time. Data were fit to the Ritger-Peppas model (solid line). D. The percentage of encapsulated insulin (2.5 mg/ml) released from PT as a function of time. Data were fit to the Higuchi model (solid line) for release of the first 60% of encapsulated insulin. Error bars represent the standard deviation from three (3) replicates.

FIG. 21. (Left) Far UV CD spectra for free insulin (0.2 mg/ml) with time over a 30 min time period following addition of chymotrypsin (0.02 mg/ml). (Right) Far UV CD spectra for insulin (0.2 mg/ml) encapsulated in phytantriol bulk cubic phase with time over a 132 min time period following addition of chymotrypsin (0.02 mg/ml).

FIG. 22. (Top) Representative far UV CD spectra for insulin (0.2 mg/ml) at 2 and 30 min after addition of chymotrypsin (0.02 mg/ml). (Bottom) Representative far UV CD spectra for insulin (0.2 mg/ml) encapsulated in phytantriol bulk cubic phase at 2, 30 and 130 min after addition of chymotrypsin (0.02 mg/ml).

FIG. 23. (Left) Near UV CD spectra for free insulin (0.5 mg/ml) with time over a 30 min time period following addition of chymotrypsin (0.05 mg/ml). (Right) Near UV CD spectra for insulin (0.5 mg/ml) in MO bulk cubic phase with time over a 132 min time period following addition of chymotrypsin (0.05 mg/ml).

FIG. 24. (Top) Representative near UV CD spectra for insulin in solution (0.5 mg/ml) at 2 and 30 min after addition of chymotrypsin (0.05 mg/ml). (Bottom) Representative near UV CD spectra for insulin (0.5 mg/ml) encapsulated in MO bulk cubic phase at 2, 30 and 130 min after addition of chymotrypsin (0.05 mg/ml).

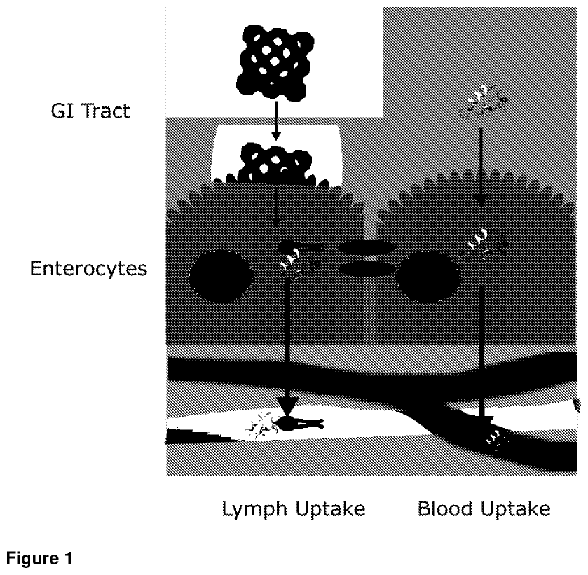

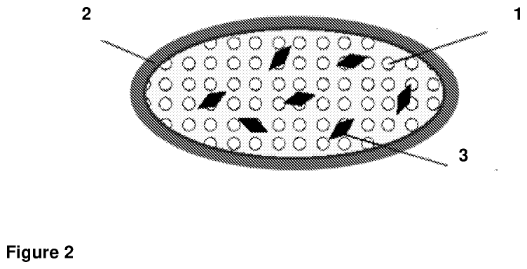

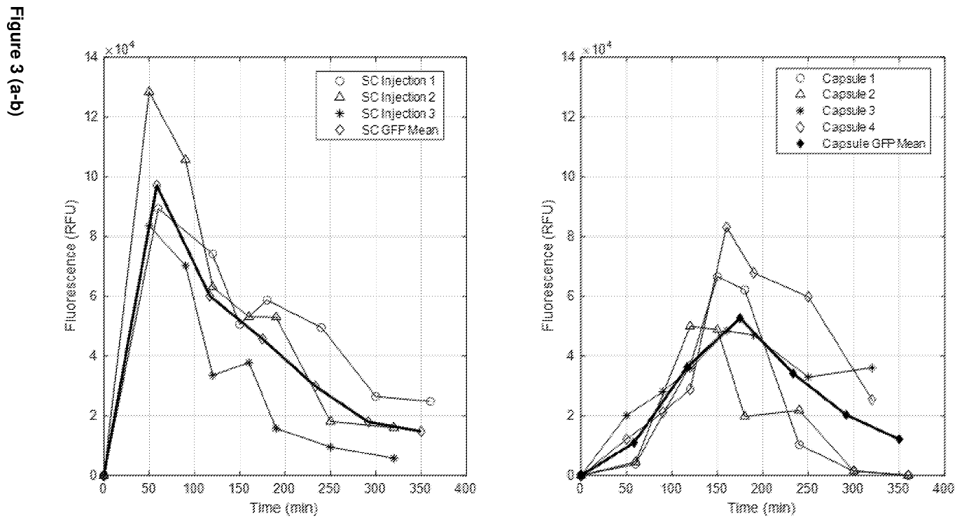

### DETAILED DESCRIPTION

Before describing the present invention in detail, it is to be understood that the terminology used herein is for the purpose of describing embodiments and examples only and is not intended to be limiting.

As used in this specification and the appended claims, the singular forms “a”, “an” and “the” include plural referents unless the content clearly dictates otherwise. Thus, for example, reference to a “lipid nanocarrier” includes a combination of two or more such lipid nanocarriers. Similarly, reference to a “therapeutic agent” includes a combination of two or more such therapeutic agents.

Throughout the description and claims of the specification the word “comprise” and variations of the word, such as “comprising” and “comprises”, is not intended to exclude other additives, components, integers or steps. As used herein, “comprises” means “includes”. Thus, “comprising A or B,” means “including A, B, or A and B,” without excluding additional elements.

Unless specifically defined otherwise, all technical and scientific terms used herein shall be taken to have the same meaning as commonly understood by one of ordinary skill in the art.

A reference herein to a document or other matter which is given as prior art is not to be taken as admission that the document or matter was known or that the information it contains was part of the common general knowledge as at the priority date of any of the claims.

As used herein, the term “therapeutic agent” as used herein refers to a drug, protein, hormone, peptide, compound or other pharmaceutically or biopharmaceutically active ingredient.

As used herein, the term “subject” shall be taken to mean any mammalian animal, preferably a human. The subject may have or be at risk of developing diabetes.

As used herein, “diabetes mellitus” refers to diabetes and related conditions including type 1 diabetes, type 2 diabetes, gestational diabetes, latent autoimmune diabetes of adulthood, maturity onset diabetes of the young, neonatal diabetes mellitus and type 3 diabetes.

As used herein, a subject “at risk” of developing a disease or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease, as known in the art and/or described herein.

As used herein, “disease”, “disorder”, “condition” and the like, as they relate to a subject's health, are used interchangeably and have meanings ascribed to each and all such terms.

As used herein, the terms “treating”, “treat” or “treatment” include administering a therapeutic agent, for example, to thereby reduce or eliminate at least one symptom of a specified disease or to slow progression of the disease.

As used herein, the term “preventing”, “prevent” or “prevention” includes providing prophylaxis with respect to occurrence or recurrence of a specified disease. An individual may be predisposed to or at risk of developing the disease or relapse but has not yet been diagnosed with the disease or the relapse.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment or prevention of a disease as described herein. In some examples of the present disclosure, the term “effective amount” is meant an amount necessary to treat or prevent diabetes mellitus. The effective amount may vary according to the disease to be treated or factor to be altered and according to the weight, age, racial background, sex, health and/or physical condition and other factors relevant to the subject being treated. Typically, the effective amount will fall within a relatively broad range (e.g., a “dosage” range) that can be determined through routine trial and experimentation by a medical practitioner. Accordingly, this term is not to be construed to limit the disclosure to a specific quantity. The effective amount can be administered in a single dose or in a dose repeated once or several times over a treatment period.

As used herein, the term “prophylactically effective amount” shall be taken to mean a sufficient quantity of the therapeutic agent to prevent or inhibit or delay the onset of one or more detectable symptoms of a disease or a complication thereof, for example inhibit or delay development of diabetes mellitus.

As used herein “oral delivery” refers to administration of a dosage form of a therapeutic agent though the oral cavity for local action or systemic absorption along the gastrointestinal (GI) tract.

As used herein, the term “gastrointestinal (GI) tract” is intended to encompass the oral cavity, oesophagus, stomach, duodenum, small intestine, large intestine (colon), rectum and anus.

As used herein “fatty acid derivate” or “a lipid derived from a fatty acid” means a formal product of a condensation reaction with a suitable functional group pendant on a head group such as a mono-, di-, or tri-substituted glycerol, glycolipid, phospholipid or ethanolamine, leading to an acyl fatty acid residue. A lipid disclosed herein can be made or obtained by any means known in the art including through both biological and synthetic means.

As used herein “dosage form” refers to any pharmaceutical preparation suitable for oral delivery of a therapeutic agent such as insulin or a derivative thereof including a pill, tablet or capsule for example.

### A Formulation for Oral Delivery of a Therapeutic Agent to a Subject

The present disclosure relates to a dosage form for oral delivery of a therapeutic agent. The therapeutic agent is first formulated into a lipid nanocarrier.

Colloidal drug carriers, such as, micelles, nanoemulsions, nanosuspensions, polymeric nanoparticles, and liposomes can overcome many issues in drug delivery such as solubility and stability. However, these systems are associated with several drawbacks, such as limited physical stability, aggregation, drug leakage on storage, lack of a suitable low-cost large-scale production method yielding a product of a quality accepted by the regulatory authorities, presence of organic solvent residues in the final product, cytotoxicity, etc.

Lipids can self-assemble to form structured bulk lipid self-assembly materials of 1-D, 2-D or 3-D symmetry. Such bulk non-dispersed lipid materials have long range order in 1D (lamellar phase), 2D (hexagonal phase) or 3D (bicontinuous cubic phase). Lipids can also form self-assembled particles of 2D symmetry (hexosomes) or 3D symmetry (cubosomes)

The present disclosure relies on structured lipid self-assembly materials as nanocarriers for the delivery of therapeutic agents. The lipid nanocarrier of the present disclosure can take the form of 2D (hexagonal phase) or 3D (bicontinuous cubic phase) symmetry. The lipid nanocarrier of the present disclosure also includes dispersed particles of 2D symmetry (hexosomes) or 3D symmetry (cubosomes)

In some examples, the lipids used to prepare the nanocarrier formulation, are physiological lipids (biocompatible and biodegradable) occurring molecules. Preferably the lipids used have low acute and chronic toxicity. The lipids may be naturally occurring.

In some examples the lipid has a chain length of C7-C35. In other examples the lipid is a long chain lipid, for example C13-C35 chain length. The lipid may be unsaturated, saturated or branched. In other examples, the lipid chain length is C13 to C19. Advantageously, it has been demonstrated that long chain lipids can be used to enhance lymphatic uptake of the therapeutic agent and by-pass first pass metabolism by the liver. This effect may be increased with increasing lipid chain length, particularly C13 to C19 chain lipids, including monoolein, phytantriol and monopalmitolein. Lymphatic transport may also increase with the degree of lipid unsaturation, such as monoolein. In some embodiments, the therapeutic agent is insulin, for example, hydrophilic insulin or a derivative thereof.

Examples of long chain lipids that can be used to prepare the formulation of nanocarrier and therapeutic include mono-, di-, or tri-substituted glycerol, glycolipid, phospholipid or ethanolamine derivatives of linear fatty acids.

Examples of such linear fatty acids include: caprylic acid, capric acid, lauric acid, myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, ceroplastic acid and docosahexaenoic acid, pelargonic acid, undecylic acid, tridecylic acid, pentadecylic acid, margaric acid, nonadecylic acid, heneicosylic acid, tricosylic acid, pentacosylic acid, carboceric acid, nonacosylic acid, hentriacontylic acid, psyllic acid, tritriacontanoic acid, ceroplastic acid, heptatriacontylic acid, nonatriacontylic acid and tetracontylic acid.

In some examples the lipid is a mono-, di-, or tri-substituted glycerol of formula I:

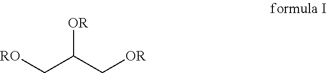

wherein at least one R is formula II, and the reaming R groups are independently selected from a hydrogen or formula II:

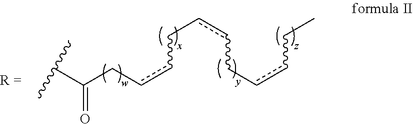

wherein w, x, y and z are independently selected from the group consisting of 0, 1, 2, 3, 4, 5, 6 and 7; wherein a broken line represents the presence or absence of a bond; and wherein a wavy bond indicates E or Z bond geometry in the presence of a bond.

In other examples, w, x, y and z are of formula II and are independently selected from the group consisting of 0, 1, 2, 3, 4, 5 and 6. In other examples w, x, y and z are of formula II and are independently selected from the group consisting of 0, 1, 2, 3, 4 and 5. In other examples w, x, y and z are of formula II and are independently selected from the group consisting of 0, 1, 2, 3 and 4. In other examples, w, x, y and z are of formula II and are independently selected from the group consisting of 0, 1, 2 and 3. In other examples, w, x, y and z are of formula II and are independently selected from the group consisting of 0, 1, and 2. In other examples w, x, y and z are of formula II and are independently selected from the group consisting of 1 and 2.

In further examples, the lipid is a mono-substituted glycerol derived from a fatty acid including the compounds of formula III:

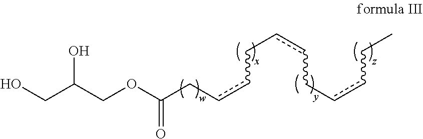

wherein w, x, y and z are selected from the group consisting of 0, 1, 2, 3, 4, 5, 6 and 7; wherein a broken line represents the presence or absence of a bond; and wherein a wavy bond indicates E or Z bond geometry in the presence of a bond.

In other examples, w, x, y and z are of formula III and are independently selected from the group consisting of 0, 1, 2, 3, 4, 5 and 6. In other examples w, x, y and z are of formula II and are independently selected from the group consisting of 0, 1, 2, 3, 4 and 5. In other examples, w, x, y and z are of formula II and are independently selected from the group consisting of 0, 1, 2, 3 and 4. In other examples, w, x, y and z are of formula II and are independently selected from the group consisting of 0, 1, 2 and 3. In other examples, w, x, y and z are of formula II and are independently selected from the group consisting of 0, 1, and 2. In other examples, w, x, y and z are of formula II and are independently selected from the group consisting of 1 and 2.

In some examples, the lipid is an unsaturated long chain lipid, for example, a long chain monoglyceride such as monoolein or monopalmitolein.

In other examples, the lipid is a branched lipid.

Examples of branched lipids include fatty acid derivatives of mono-, di-, or tri-substituted glycerol, glycolipid, phospholipid or ethanolamine derivatives. Such fatty acids include mycolipanolic acid, mycoceranic acid, mycolipenic acid, micolipodienoic acid, mycocerosic acid, phthioceranic acids, dolichoic acids, phytanic acid, pristanic acid, from branched hydroxy fatty acids (mycolic acids), methoxymycolic acids, ketomycolic acids, 1-monomethyl branched fatty acids, 1-methyloctadec-12-enoic and 12-methyloctadec-10-enoic acids, cis-11-methyl-2-dodecenoic acid, tuberculostearic acid, phytomonic acid, 7-methyl-6-octadecenoic and 17-methyl-7-octadecenoic acids and laetiporic acid.

Further examples of branched lipids relate to multi-branched lipids including isoprenoid-like lipids such as phytantriol and those derived from retinoic acid.

In further examples, the lipid is a charged lipid. Examples of charged lipids include phospholipids such as glycerophospholipids including phosphatidates, phosphatidylserine, phosphoinositides, phosphatidylinositol, phosphatidylinositol phosphate, phosphatidylinositol bisphosphate, phosphatidylinositol trisphosphate and phosphosphingolipids.

In other examples the phospholipid lipid can be phosphatidylethanolamine or phosphatidylcholine.

Further examples of charged phospholipids include sphingolipids such as ceramide phosphorylcholine, ceramide phosphorylethanolamine and ceramide phosphoryllipid.

In some examples, the lipid is a glycolipid lipid. Examples of glycolipids include glyceroglycolipids, galactolipids, gangliosides, globosides, glycophosphosphingolipids and glycophosphatidylinositols.

In other examples, the formulation of lipid nanocarrier comprises one of more of the lipids as described.

### Lipid Nanocarrier Structure

Lipids can form different structures such as lyotropic liquid crystalline

phases when mixed with an aqueous solution, typically water, which can be distinguished, for example, by their characteristic small angle X-ray scattering patterns, particle size, zeta potential and polydispersity index.

The term “mesophase” is used to indicate the distinctive self-assembled structure between a liquid and solid crystal phase. A liquid phase is fluid, while an ordered crystalline structure defines the solid state. The different mesophases include the cubic phase, hexagonal phase, lamellar phase, micellar, in both continuous and dispersed forms. Other possible forms of liquid crystalline phases include inverse hexagonal (H2), bicontinuous inverse cubic (V2) including primitive (Im3m), double diamond (Pn3m), gyroid (Ia3d), reverse cubic micelles (I2), sponges (L3), inverse micelles (L2), disordered micellar, micellar, vesicles (Lα), lamellar (Lα) and liposomal.

The self-assembly of lipids is tuneable by varying formulation conditions

such as temperature, lipid concentration, homogenisation and through the addition of modifiers and stabilisers. This allows for mesophases to assemble lipid nanoparticles of varying morphology. Often the morphology leads to three dimensional networks of lipids and solvent channels which can be tuned to accommodate different therapeutic agents and have differing properties such as dissolution profiles.

Tuning of dissolution profiles can lead to delayed release allowing for targeting release of the therapeutic agent in particular areas of the gastrointestinal tract. For example, the duodenum specifically can be targeted with Eudragit L100-55 while the upper small intestine can be targeted with Eudragit L 100. Lipid nanocarriers in the form of a mesophase comprising therapeutic agents, such as insulin, have been shown to offer protection against enzymatic degradation in systems that mimic in vivo gastrointestinal environments. Such environments include at least 2 hours after addition of the chymotrypsin enzyme. In contrast, the complete degradation of insulin in water was observed over less than 35 min in the absence of a protective matrix. This work demonstrates that the lipids in the form of a mesophase are effective at protecting encapsulated therapeutic agents against degradation by digestive enzymes.

Without wishing to be bound by theory, aqueous lipid nanocarriers having the form of a mesophase that comprise therapeutic agents provide further protection from gastrointestinal environments. Such dosage forms avoid hydration in the gut and thus exposing the therapeutic agent to gastrointestinal environments to various lipases and peptidases, including chymotrypsin. Moreover, by avoiding gut hydration, it is theorized that mesophasic lipid nanocarriers comprising a therapeutic agent exit from the gut though the lymphatic system and as such to avoid the circulatory system and first pass metabolism, thus improving bioavailability and biodistribution.

The therapeutic agent and lipid can be formulated into a mesophase by any known means in the art including mixing for example, in a syringe, cold or hot high-pressure homogenization, emulsification-sonification, solvent emulsification-evaporation, solvent diffusion, microemulsion, solvent injection and/or double emulsion.

### Cubic Phase

In some examples, the present disclosure relates to the lipid nanocarrier taking the form of a cubic phase. The cubic phase being a non-dispersed lipid nanocarrier formed via the self-assembly of lipids and having long range order in three (3) dimensions.

Bicontinuous Cubic Phase

In further examples, the lipid nanocarrier is in the bicontinuous cubic phase which is arranged in a pattern of infinite periodic minimal surfaces (IPMSs). This is further divided into primitive (Im3m), double diamond (Pn3m) and gyroid (Ia3d) phases.

The bicontinuous cubic mesophase forms a three-dimensional network of lipid bilayers separated by two water channels. Formulation of a nanocarrier in this phase can incorporate therapeutic agents of varying physicochemical properties.

In some examples, the bicontinuous cubic mesophase can be formulated using unsaturated or long-chain monoglycerides, such as monoolein or monopalmitolein, or long-chain branched lipids, such as phytantriol; and water at varying concentrations resulting in tuneable properties including stability and dissolution profile in the digestive tract, for example, in the small intestine.

In some examples, the bicontinuous cubic phase formulation has a lipid concentration of 10% to 95% weight of the formulation. In other examples the nanocarrier has a lipid concentration of 25% wt to 85% wt, in other examples the nanocarrier has a lipid concentration of 35% wt to 80% wt, in other examples the nanocarrier has a lipid concentration of 55% wt to 75% wt.

In some examples the bicontinuous cubic phase formulation is aqueous and has a water content at room temperature is as follows:

| Cubic Phase | From | To |
|---|---|---|
| Water(%) weight of the formulation | 1 | 70 |
| Water(%) weight of the formulation | 20 | 60 |
| Water(%) weight of the formulation | 30 | 50 |
| Water(%) weight of the formulation | 36 | 42 |

In a particular example of the bicontinuous cubic phase, the water content is about 38% weight of the formulation.

In more specific examples, the reverse (or inverse) form of the bicontinuous cubic phase comprises lipid concentrations of monoolein of about 40% to 80% weight of the total formulation. In other examples, the monoolein is present in 50% to 65%, 50% to 64%, 60% to 63% weight of the total formulation. In specific examples the monoolein content is greater than or equal to 52% weight of the total formulation. In these examples and where the bicontinuous cubic phase formulation is aqueous, the formulation has a water content at room temperature of up to and including 48% weight of the formulation. In other examples the water content is 1% to 48%, 20% to 44%, 30% to 42% or 36% to 42% weight of the formulation. In a particular example, the water content is about 38% weight of the formulation.

In more specific examples, the reverse (or inverse) form of the bicontinuous cubic phase comprises lipid concentrations of phytantriol of about 60% to 75% weight of the total formulation. In other examples, the phytantriol is present in 65% to 75%, 68% to 74%, 70% to 73% weight of the total formulation. In a specific example the phytantriol content is 72% weight of the total formulation. In these examples and where the bicontinuous cubic phase formulation is aqueous, the formulation has a water content at room temperature up to and including 28%. In other examples the water content is 1% to 28%, 20% to 25%, 22% to 36% weight of the formulation.

In other examples the reverse (or inverse) form of the bicontinuous cubic phase comprises a combination of the lipids as described.

The water channel size may be, for example, between 1 nm to 17 nm. For example, the aqueous channel size can be from 1 nm to 7 nm. In further examples, the cubic mesophase can be fragmented into dispersions of cubic particles using an adequate surfactant. Such surfactants include, for example, Poloxamer 407.

Cubosomes

In some examples the lipid nanocarrier takes the form of cubosomes. Cubosomes are dispersed, sub-micron, nanostructured particles having three (3) dimensional symmetry.

Cubosomes can be formulated to specific pore sizes to incorporate therapeutic agents using methods known in the art. Their structure provides a high surface area for loading of therapeutic agents.

In some examples, stabilisers can be added to stabilise the structure of the nanocarrier when in the form of a cubosome.

Examples of stabilisers include, for example, poloxamer 407, polyethylene glycol, pluronic F108, F68, F38, F127, F87NF, P105, P85, L35, P104, P84, L64, P123, P103, L43, L92, L62, L121, L101, L81 and L61.

Stabilisers can be present in a range of 0.01% to 10% weight of the lipid present, for example, 5% to 10% weight of the lipid. In some examples, the stabiliser is present at 10% weight of the lipid. In these examples and where the formulation is aqueous, the formulation has a water content at room temperature of up to and including 48% weight of the formulation. In other examples the water content is 1% to 48%, 20% to 40%, 25% to 35%, 38% to 40% weight of the formulation.

Hexagonal Phase

In some examples, the present disclosure relates to the lipid nanocarrier taking the form of a hexagonal phase. The hexagonal phase being a non-dispersed lipid nanocarrier formed via the self-assembly of lipids and having long range order over two (2) dimensions.

In some examples the lipid used to form the hexagonal phase is di-oleoyl phosphatidylethanolamine (DOPE).

In particular examples the present disclosure relates to the lipid nanocarrier taking the form of a reverse hexagonal phase (HII).

Hexosomes

In some examples the lipid nanocarrier takes the form of hexosomes. Hexosomes are dispersed, sub-micron, nanostructured particles having two (2) dimensional symmetry.

In some examples the lipid nanocarrier takes the form of hexosomes. Hexosomes are reverse hexagonal phases comprised of hexagonally close-packed infinite water layers covered by a surfactant monolayer.

Hexosomes can be formulated with therapeutic agents by, for example, accommodation within the water layers.

Example of surfactants include amino acid-based catanionic surfactants such as arginine-N-lauroyl amide dihydrochloride (ALA) and N-lauroyl-arginine-methyl ester hydrochloride (LAM) with two and one positive charges per headgroup, respectively, and sodium hydrogenated tallow glutamate (HS).

Further examples of surfactants include anionic surfactants such as sodium octyl sulphate (SOS) and sodium cetyl sulphate (SCS).

Additional modifiers can be present in the formulation of hexosomes include oleic acid, tetradecane and vitamin E acetate.

In some examples, stabilisers as described can be added to stabilise the structure of the nanocarrier when in the form of a hexosome.

### Solid Lipid Nanoparticles

In some examples, the lipid nanocarrier takes the form of nanoparticles, which include nanospheres and nanocapsules, dendrimers, solid lipid nanoparticles, transfersomes and nanogels.

Solid lipid nanoparticles (SLNs) are often prepared from lipids which are solid at room temperature as well as at body temperature. Solid lipid nanoparticles can protect therapeutic agents which are photosensitive, moisture sensitive, and chemically labile. Typically, SLN dispersions contain a high amount of water.

Examples of lipids that can form solid lipid nanoparticles include tripalmitin, cetyl alcohol, cetyl palmitate, Compritol 888 ATO, glyceryl monostearate, precirol® ATO5, trimyristin, tristearin, stearic acid and Imwitor®900.

### Excipients, Diluents, Adjuvants, Swelling Agents and Modifiers

The lipid nanocarrier formulation may further comprise one or more of excipients, diluents, adjuvants, swelling agents, modifiers and/or stabilisers.

For example, the formulation may comprise one or more of charged lipids, branched lipids, carboxylic acids, ionic surfactants, polyelectrolytes, water-soluble surfactants, water-insoluble surfactants, hydrophilic cosolvents, and/or small molecules.

In specific examples, the formulation may comprise one or more of charged lipids, branched lipids, carboxylic acids such as oleic acid.

Specific examples include, pyridinylmethyl linoleate, 2-hydroxyoleic acid, oleic acid, pluronic F127, phloroglucinol, N-Oleoyl-glycine, N-(2-aminoethyl)-oleamide, vaccenic acid, oleic acid, gondoic acid, erucic acid and nervonic acid.

In some examples, the formulation comprises one or more cationic lipids such as dioleoyl-3-trimethylammonium propane (DOTAP) and 1,2-dioleoyl-3-trimethylammonium-propane. Such cationic lipids can be present in a range of, for example, 1% to 10% weight.

In some examples DOTAP is present in an amount of up to an including 10% weight of the lipid nanocarrier formulation. In other examples, DOTAP is present at 0.1% to 15%, 1% to 10%, 4% to 10% or 6% to 10% weight of the lipid nanocarrier formulation.

In some examples, the formulation comprises cholesterol which is typically used as a swelling agent for swelling of the cubic phase. The swelling agent, such as cholesterol can be present up to, for example, up to 50% by weight.

In some examples the lipid nanocarrier formulation is swollen, particularly to accommodate large proteins, the water content is up to and including 70% weight of the lipid nanocarrier formulation. In further examples the water content is 48% to 70%, 50% to 65% or 60% to 65% weight of the formulation.

### Enteric Coating

In some examples, following formulation of the therapeutic agent into a lipid nanocarrier, the formulation can be encapsulated in an enteric coating to provide a dosage form.

In a further set of examples, the enteric coating can be applied to a capsule

shell and a capsule filling comprising the formulation. In examples where the coating is applied to a shell, the shell can be comprised of any suitably soluble material that is soluble in the GI environment, particularly the small intestine.

Examples of enteric coatings that can be used include, for example, those shown in table 1:

| | | | Dissolution |
|---|---|---|---|
| | Product | Availability | property |
| | Eudragit ® L 100-55 | Powder | Dissolution above |
| RTU | Acryl-EZE ® | Ready-to-use | pH 5.5 |
| | (functional polymer: | colour matched | |
| | Eudragit ® L 100-55) | powder mixture | |
| | Eudragit ® L 30 D-55 | Aqueous | |
| | | dispersion | |
| ETU | PlasACRYL ™ HTP20 | Easy-to-use | — |
| | | glidant and | |
| | | plasticizer premix, | |
| | | specifically | |
| | | designed for | |
| | | Eudragit L 30 D-55 | |
| | | formulations | |
| | Eudragit ® L 100 | Powder | Dissolution above |
| | Eudragit ® L 12.5 | Organic solution | pH 6.0 |
| | Eudragit ® s 100 | Powder | Dissolution above |
| | Eudragit ® s 12.5 | Organic solution | pH 6.0 |
| | Eudragit ® FS 30 D | Aqueous | |
| | | dispersion | |
| ETU | PlasACRYL ™ T20 | Easy-to-use | |
| | | glidant and | |
| | | plasticizer premix, | |
| | | specifically | |
| | | designed for | |
| | | Eudragit ® FS 30 D | |
| | | formulations | |

In some examples the enteric coating is soluble at a range of about pH 5.6 to pH 7.2, in further examples the enteric coating is soluble at range of about pH 5.8 to pH 6.5. In further examples the enteric coating is soluble at range of about pH 4.5 to pH 7.2, In further examples the enteric coating is soluble at range of about pH 5.0 to pH 6.0.

In some examples, the enteric coating has a thickness in a range of 0.01 nm to 1.00 mm. In other examples the range is 10 μm to 500 μm, in further examples the range is 100 μm to 200 μm.

In some examples, where the dosage form is encapsulated in the enteric coating, the coating has a thickness in a range of 0.05 μm to 1 mm, 1 μm to 0.5 mm, 10 μm to 0.1 mm or 50 μm to 0.5 mm. In a specific example, the range is 0.07 mm to 0.4 mm.

In some examples the dosage form is a capsule comprising a filling comprising the lipid nanocarrier formulation and a shell encapsulating the filling, the shell coated in the enteric coating.

In some examples, the enteric coating is applied to at least one of the shell surface facing the filling and the outer shell surface. In those examples the shell surface facing the filling and the outer shell surface each independently have a thickness in a of range of 30 μm to 500 μm. In other examples the range is 200 μm to 400 μm, in further examples the range is 80 μm to 180 μm

In examples where the lipid nanocarrier is aqueous having thicker enteric coatings can aid in stability of the dosage form ex vivo.

Further examples of the thickness of the enteric coating are provided in table 2:

| | Example of upper and | Particular example of |
|---|---|---|
| | lower limits on | thickness of the enteric |
| Location of coating | thickness | coating |
| Range of thickness of | 30 μm-380 um | 80 μm-180 μm |
| enteric coating on an | | |
| outer shell surface | | |
| Range of thickness of | 30 μm-380 um | 80 μm-180 μm |
| enteric coating on a | | |
| shell surface facing the | | |
| filling | | |
| Range of thickness of the | 0.05 μm-1 mm | 0.07 mm-0.4 mm |
| coating if the dosage | | |
| form is encapsulated in | | |
| the enteric coating | | |

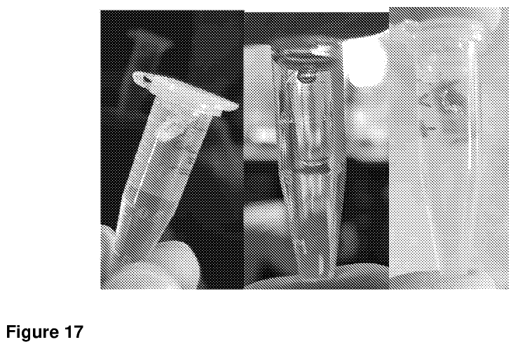

FIG. 17 on the left-hand side shows that a double enteric (Eudragit L 100) coated capsule is still stable after 24 hours in pH 4 media inside and outside. The middle section of this figure shows a standard capsule empty at t=0. On the right-hand side is shown a single outside coating with pH 4 media inside and outside, showing destruction of the capsule after 8 hours. In some examples, it is theorized that an inner enteric coating can prevent a hydrated cubic phase from coming into contact with the capsule and aid in preventing ex vivo degradation.

### Therapeutic Agents

The present disclosure relates to a formulation for oral delivery of a therapeutic agent.

The therapeutic agent can be dispersed, contained, conjugated, and/or absorbed within the lipid nanocarrier. Furthermore, the therapeutic agent can be incorporated in the lipid nanocarrier by any means such as a homogenous matrix, enriched on a surface of the lipid nanocarrier, within a membrane and/or within a cavity of the lipid nanocarrier.

In some examples, the therapeutic agent is a peptide, for example, insulin.

The insulin or derivates thereof may be long-, ultralong- or intermediate- acting insulin. Examples of these insulins include glargine (Lantus, Basaglar, Toujeo), detemir (Levemir), degludec (Tresiba) and NPH (Humulin N, Novolin N, Novolin ReliOn Insulin N). Alternatively, the insulin may be rapid- or short-acting insulin. These insulins are ideal for preventing blood sugar spikes after you eat. Examples of these insulins include aspart (NovoLog, Fiasp), glulisine (Apidra), lispro (Humalog, Admelog) and regular (Humulin R, Novolin R).

In other examples, the peptide is a neuropeptide, for example, somatostatin (SST) also known as growth-hormone inhibiting hormone (GHIH) or oxytocin.

Other therapeutic agents include steroidal hormones. Examples of steroidal hormones include glucocorticoids, mineralocorticoids, androgens, estrogens and progesterones.

In further examples, the therapeutic agent is a protein, for example, a protein up to 170 kDa in size.

In some embodiments, the therapeutic agent is between 5 and 150 kDa, between 5 and 100 kDa, between 5 and 50 kDa, between 5 and 40 kDa, between 5 and 30 kDa, between 5 and 20 kDa, or between 5 and 10 kDa in size. In one embodiment, the therapeutic agent is about 5 kDa in size.

In some examples the protein is an hormonal protein such as human growth hormone or human coagulation factor X. In some examples the therapeutic agent is a small molecule. For example, the small molecule can be an antibiotic or antimicrobial. Such antibiotics include glycopeptide peptides such as vancomycin or β-lectern antibiotics such as meropenem.

Oral administration of these therapeutics is often ineffective due to chemical and/or enzymatic degradation in the gastrointestinal tract. Current modes of administration of such therapeutic agents include intravenous, intramuscular and/or sub cutaneous injection due to the lack of bioavailability of oral administration. These modes of administration are often accompanied by sometimes serve side effects associated with the injection.

In examples where the nanocarrier comprises lipids in the bicontinuous cubic phase, insulin derivatives can be present in 0.01% to 1% weight of the formulation. In other examples insulin derivatives can be present in 0.05% weight to 0.5% weight of the formulation. In further examples, insulin derivatives can be present in 0.1% weight to 0.3% weight of the formulation.

### A method for preparing a Capsule

In some examples, the dosage form can be a capsule and can be made accordingly by the following steps, providing the capsule filling comprising the formulation as described and encapsulating the capsule filling in an enterically coated shell or encapsulating the dosage form directly with an enteric coating.

The formulation as described can be prepared by contacting the lipid, the therapeutic agent and an aqueous solvent under conditions sufficient to induce nanocarrier formation.

In other examples, the formulation can be formed by contacting the lipid, aqueous solvent and therapeutic agent using the following methods known in the art: mixing for example in a syringe, cold or hot high-pressure homogenization, emulsification-sonification, solvent emulsification-evaporation, solvent diffusion, microemulsion, solvent injection and/or double emulsion.

Examples of contacting the lipid in an aqueous solution can be at a lipid concentration of 10% to 90% weight of the solution. In examples where the formulation is aqueous, the formulation has a water content at room temperature of 1% to 70%, 20% to 60%, 30% to 50% or 36% to 42% weight of the formulation.

In particular examples related to swollen cubic phases, the lipid concentration is 10% to 50%. In these examples, the water content is up to and including 70% weight of the lipid nanocarrier formulation. In further examples the water content is 48% to 70%, 50% to 65% or 60% to 65% weight of the formulation

In some examples, the formulation is prepared by containing the lipid, the therapeutic agent and aqueous solvent to give multiphasic mixture. This mixture is then introduced to a first mixing chamber connected to a second mixing chamber by way of a mixing attachment adapted for homogenization. The mixture is passed through the mixing attachment adapted for homogenization until the inverse bicontinuous cubic phase is formed.

In a specific example, the ratio of the lipid:aqueous solvent is about 60:40 w/w, and the first and second mixing chambers is a syringe, passage through the mixing attachment adapted for homogenisation can be conducted up to 50 times.

Formulation can be conducted at 20° C. to 90° C. In other examples, the temperature is in a range of 20° C. to 50° C. In other examples, the temperature is in a range of 20° C. to 35° C.

In some examples the capsule filling comprising the formulation of lipid nanocarrier and therapeutic agent can be inserted into a pre-prepared shell coated with an enteric coating. In other examples, the enterically coated capsule shell is applied to the filling comprising the formulation of lipid nanocarrier. Application of the enteric coating can be conducted by any means suitable including dipping or spraying.

In further examples the enteric coating is applied to at least one of the shell surface facing the filling and the outer shell surface.

In a particular example, a capsule, for example a gelatin capsule, can be coated with enteric coating, such as by use of a dipping tray, creating the enteric coated capsule shell. Capsules can be dipped for example, up to three times and left to dry for about 15 min.

The enterically coated capsule shell can then be filled with the formulation comprising the lipid cubic phase and therapeutic agent. Alternatively, two halves of a capsule can be joined to encapsulate the capsule filling comprising the formulation of lipid nanocarrier and therapeutic agent.

For industrial scales, spray coating of enteric capsules could be used when scaling up to a large number of capsule shells.

In particular examples the therapeutic agent of the dosage form is an insulin, an antibiotic, or a protein hormone present in a therapeutically or prophylactically effective amount. The mesophase can be cubic, primitive cubic phase (Pn3m), diamond cubic phase (Ia3d) or gyroid cubic phase, or inverse hexagonal phase the mesophase is aqueous with a water content of 0.1% to 48% weight of the formulation, or a mixture of phases. The lipid can be long chain lipid such as monoolein, phytantriol or monopalmitolein present in an amount of 35% to 62% weight of the formulation. The enteric coating can be present at a thickness of 160 μm to 500 μm. Without wishing to be bound by theory, an inner enteric coating can prevent the hydrated cubic phase from coming into contact with the capsule and aid in preventing degrading it ex vivo.

### Methods of Treatment

Oral administration of therapeutic agents, including insulin, is preferred because of its convenience, the relatively low production cost and the high level of patient safety. However, a considerable proportion of therapeutic agents do not display the characteristics required for oral administration.

A prerequisite for a therapeutic agent to be efficient after oral administration is that it largely avoids a sequential series of barriers in the GI tract and in the liver. Systemic bioavailability of orally administered therapeutic agents has primarily been considered to be a function of intestinal drug absorption and subsequent phase I metabolism in the liver. However, the human small intestine has increasingly been recognised as an important site for first pass extraction.

Therapeutic agents designed to be systemically active must be absorbed from the site of administration in order to be efficient. Furthermore, to allow passage through the biological membranes, the therapeutic agent must be in solution. Since most therapeutic agents are administered as solid dosage forms, disintegration of the formulation must precede dissolution of the therapeutic agent in the surrounding media. The disintegration rate is influenced by characteristics of the formulation and by physiological factors such as gastric emptying rate, the transit time and the pH of the gastrointestinal fluids. Once in solution, the therapeutic agent is susceptible to both chemical and enzymatic degradation. The bioavailability may be further reduced by efflux mechanisms or first-pass metabolism in the intestinal epithelium and/or the liver.

Surprisingly, it has been demonstrated that formulation of a therapeutic agent in a lipid nanocarrier that is then encapsulated in an enteric coating allows for oral absorption and systemic bioavailability of the therapeutic agent. It has further been demonstrated improved oral absorption and bioavailability of insulin that is comparable to insulin administered systemically. As such, the insulin dosage form of the present disclosure can be used to treat or prevent diabetes mellitus. Methods of the disclosure comprise orally administering such dosage form to a subject in need to treat or prevent diabetes mellitus.

Diabetes mellitus (DM) is a chronic metabolic illness which is estimated to affect 451 million individuals at the present date, with this number expected to significantly rise in the coming years. Diabetes is characterized by sustained hyperglycaemia, which over time results in diabetic complications and eventually death. Blood glucose levels (BGLs) above 7.0 mmol/L during fasting and 11.1 mmol/L postprandial are indicative of diabetes.

Insulin therapy is pivotal in the management of diabetes, with diabetic individuals taking multiple daily insulin injections. However, the mode of administration has numerous drawbacks, resulting in poor patient compliance.

In some examples, the dosage form of the disclosure can be used for oral administration of insulin or a derivative thereof for the treatment or prevention of diabetes mellitus.

Specific embodiments and applications of the present disclosure will now be discussed in detail by reference to the accompanying examples. This discussion is in no way intended to limit the scope of the disclosure.

The dosage form as described can further find use in the treatment of diabetes mellitus, a condition mediated by bacterial infection, a condition mediated by human growth hormone, or a condition mediated by blood coagulation and where the therapeutic agent is present in a therapeutically or prophylactically effective amount.

The dosage form as described can be used in a method for treating or preventing diabetes mellitus, a condition mediated by bacterial infection, a condition mediated by human growth hormone, or a condition mediated by blood coagulation which comprises administering to a subject in need the dosage form as described and the therapeutic agent is present in a therapeutically or prophylactically effective amount.

Further uses of the dosage as described is for the treatment or prevention of diabetes mellitus, a condition mediated by bacterial infection, a condition mediated by human growth hormone, or a condition mediated by blood coagulation and the therapeutic agent is present in a therapeutically or prophylactically effective amount.

Examples of a condition mediated by bacterial infection include gram-positive pathogens, infection caused by methicillin-resistant S. aureus, infection caused by multidrug-resistant S., epidermidis, endocarditis, primary sclerosing cholangitis endophthalmitis, gram-negative pathogens, urinary tract infections, meningitis, intra-abdorninal infection, pneumonia, sepsis, or anthrax.

Examples of a condition mediated by human growth hormone include growth hormone deficiency such as, in childhood, in adulthood, AIDS wasting, renal failure, turner syndrome, achondroplasia, Prader-Willi syndrome, poor growth in children small for gestational age or idiopathic short stature.

Examples of a condition mediated by blood coagulation include to treat or prevent bleeding in people with hereditary factor X deficiency.

### EXAMPLES

### Example 1. Materials and Methods

Monoolein (oleoyl-rac-glycerol)(>99%) was purchased from Sigma Aldrich. Green fluorescent protein (GFP) (>99%) and red fluorescent protein (RFP) (>99%) were sourced from Biovision. Oral gavage and size 9 capsules used in the animal trials were sourced from Torpac, with Eudragit L 100 sourced from Evonik. Accu Chek BG monitor and strips were purchased from Priceline pharmacy. ActRapid and Levemir (Novo Nordisk) were purchased at Chemist Warehouse.

Formulation of the Lipid Nanocarrier in the Cubic Phase

For the preparation of the lipid nanocarrier in the cubic phase with GFP encapsulated, a known amount of monoolein (typically 50 mg) was added to a 100 μl syringe. A solution of GFP in PBS (4 mg/ml) was added to another 100 μl syringe in the ratio 60:40 w/w lipid: protein solution. The contents of both syringes were mixed using a specialized syringe mixing attachment.

For the preparation of the lipid nanocarrier in the cubic phase comprising Actrapid insulin, a known amount of monoolein (typically 50 mg) was added to a 100 μl syringe. A solution of Actrapid insulin in PBS (100 IU/ml) was added to another 100 μl syringe in the ratio 60:40 w/v lipid: protein solution. The contents of both syringes were mixed using a specialized syringe mixing attachment.

For the preparation of the lipid nanocarrier in the cubic phase comprising Levemir insulin, a known amount of monoolein (typically 50 mg) was added to a 100 μl syringe. A solution of Actrapid insulin in PBS (100 IU/ml) was added to another 100 μl syringe in the ratio 60:40 w/w lipid: protein solution. The contents of both syringes were mixed using a specialized syringe mixing attachment.

The specialised mixer consists of two 100 μL (Hamilton Company, cat #7656-01) or two 250 μL (Hamilton Company, cat #7657-01) gas-tight syringes and a syringe coupler made of two removable needle (RN) nuts (Hamilton, cat #30902) and two gauge 22 removable needles (Hamilton, cat #7770-02) (Cheng et al., 1998). Alternatively, a slightly different syringe coupler can be purchased from Emerald Biosystems (cat #EB-LCP-SUNION), or from Molecular Dimensions (cat #MD6-17). The lipid mixer allows for fast and efficient mixing of small volumes of lipids and aqueous solutions (total volume of 10-100 μL with 100 μL syringes, and 25-250 μL with 250 μL syringes).

Enterically Coated Capsule Preparation

A diluent mixture was created using 342.9 g Acetone, 514.2 g Isopropanol and 42.9 g of Milli Q water and poured into a 3L beaker. A high torque mixer was used to mix 62.5 g of Eudragit L 100 into the diluent suspension and was slowly mixed for 60 minutes. 6.25 g of Triethyl citrate was added and stirred for a further hour before being passed through a 0.5 mm sieve to create the Enteric Coating mixture. Capsules were coated in this mixture using a dipping tray. For the GFP trial, each side of the capsule was dipped briefly two times and dried (Thickness T=2). For the first phase of the insulin trial, each side of the capsule was dipped briefly three times and dried (T=3). For the second phase of the insulin trial, each side of the capsule was dipped briefly once and dried (T=1).

These capsules were then filled with 25 μl of the GFP loaded LCP as described above. The final amount of GFP in each capsule was 50 μg. For delivery of insulin, capsules were filled with 25 μl of the insulin loaded LCP as described above this. The final amount of insulin in each capsule was 1 IU.

Phase 1 trial. Each capsule was dipped three times−coating thickness=3 layers of Eudragit L 100 enteric coating.

Phase 2 trial. Each capsule was dipped once−coating thickness=1 layer of Eudragit L 100 enteric coating.

Preparation of an Enterically Coated Formulation of Insulin and Monoolein in the Cubic Phase

A diluent mixture was created using 342.9 g Acetone, 514.2 g Isopropanol and 42.9 g of Milli Q water and poured into a 3L beaker. A high torque mixer was used to mix 62.5 g of Eudragit L 100 into the diluent suspension and was slowly mixed for 60 min. 6.25 g of Triethyl citrate was added and stirred for a further hour before being passed through a 0.5 mm sieve to create the enteric coating mixture. Gelatin capsules were coated in this enteric mixture. Tweezers were used to dip one half of each capsule into the enteric mixture, followed by air drying over 15-20 mins on tissue paper. The second half of the capsule was then dipped and also air dried over 15-20 mins on tissue paper. The film coating thickness was increased by dipping one, two, or three times, with air drying in between.

For the preparation of lipidic cubic phase with Actrapid insulin dispersed, a known volume of molten monoolein (50 μl) was added to a 100 μl syringe. A solution of Actrapid insulin in PBS (40 μl, 100 IU/ml) was added to another 100 μl syringe. The contents of both syringes were mixed using a specialized syringe mixing attachment. The resulting mixture was visually observed to be cloudy due to the presence of excess water. To ensure the cubic phase was formulated at just under excess water conditions a small amount of lipid was subsequently added and mixed using the syringe mixer until the formulated cubic phase was visually observed to be clear with no evidence of any cloudiness.

The insulin dispersed lipid cubic phase created in step 2 was injected directly from the 100 μl syringe into the capsules created in step 1. To do this the syringe mixing attachment was replaced with a high-gauge needle.

### Example 2: Animal Trial 1 (GFP)

GFP/RFP Pre-Trial

Two Sprague Dawley rats were used in this part of the trial. Each was initially given isoflurane until unconscious. This was followed by the preparation of the saphenous vein and the first blood collection (100 μl). Rat 1 was then subcutaneously injected with 500 μl of 100 μg/ml GFP while Rat 2 was subcutaneously injected with 500 μl of 100 μg/ml RFP. The animals were then housed separately in two boxes. Blood collection (100 μl) was repeated every half hour for 120 minutes then every hour until the end of the trial at 6 hours. At the end of the trial blood was separated using a centrifuge at 1200 rpm for 2 min. Following this, the blood plasma supernatant was removed and frozen at −40° C. for fluorescence analysis on a CLARIOstar microplate reader (BMG Labtech) the following day.

GFP (Subcutaneous Injection)

Three Sprague Dawley rats were used in this part of the trial. Each was initially given isoflurane until unconscious. This was followed by preparation of the saphenous vein and the first blood collection (100 μl). The rats were then subcutaneously injected with 500 μl of 100 μg/ml GFP before being housed separately in three boxes. Blood collection (100 μl) was repeated every half hour for 120 minutes then every hour until the end of the trial at 6 hours. At the end of the trial blood was separated using a centrifuge at 1200 rpm for 2 minutes. Following this, the blood plasma supernatant was removed and frozen at −40° C. for fluorescence analysis on a CLARIOstar microplate reader (BMG Labtech) the following day.

GFP (Oral Capsule)

Four Sprague Dawley rats were used in this part of the trial. Each was initially given isoflurane until unconscious. This was followed by the preparation of the saphenous vein and the first blood collection (100 μl). An enteric capsule (T=2) containing GFP loaded cubic phase (25 μl of cubic phase containing 50 μg GFP) was then introduced via oral gavage into the rat stomach. The animals were then housed separately in four boxes. Blood collection (100 μl) was repeated every hour for 120 minutes, then every 30 minutes until 180 minutes, then every 60 minutes until the end of the trial at 6 hours. At the end of the trial blood was separated using a centrifuge at 1200 rpm for 2 minutes. Following this the blood plasma supernatant was removed and frozen at −40° C. for analysis the following day.

Fluorescence Analysis

Fluorescence analysis of blood plasma samples was carried out on a CLARIOstar microplate reader (BMG Labtech). 2 μl of blood plasma from each time point was thawed for 5 minutes and contained within an LVis-nanoplate and run in fluorescence mode at the standard GFP excitation and emission wavelengths (488/510 nm). All measurements were taken at 25° C.

### Example 2: Animal Trial 2 (Insulin)

Diabetes Induction via STZ Injection

14 Sprague Dawley rats were used in this trial. One week before each phase started, all rats were given isoflurane until unconscious. Each rat was then injected intravenously in the tail with 60 mg/kg of streptozotocin (STZ) in a sodium citrate buffer. All rats were then housed in pairs and blood glucose was monitored 3 times per day over 48 hours. The induction of diabetes was confirmed when the blood glucose level was >14 mmol/L in the morning.

At this point, 15 IU fast-acting (Actrapid) insulin was administered via SC injection as required until the start of the trial. The rats were then housed in pairs in two boxes. Blood collection was taken at 5-, 360-, and 1440-minutes post-injection, and the BG level tested at each time point. This data was used to confirm that each rat was diabetic prior to the commencement of the trial and to determine the average blood glucose increase in rats in the absence of any treatment.

Delivery of Fast Acting Insulin (Actrapid) by Subcutaneous Injection (Phases 1 and 2)

Four Sprague Dawley rats were used in Phase 1 and four in Phase 2. Each was initially given isoflurane until unconscious. This was followed by preparation of the saphenous vein and the baseline basal BG was tested. The rats were then subcutaneously injected with 1 IU fast-acting insulin (Actrapid), immediately following this they were placed on a heat-mat until they regained consciousness, before being housed in pairs in boxes. Blood collection (10 μl) was taken at 15-, 30-, 60-, 120-, and 360-minutes post-injection. BG testing was performed immediately at each time point using an Accu Chek BG monitor and strips.

Delivery of Slow Acting Insulin (Levemir) by Subcutaneous Injection (Phases 1 and 2)

Four Sprague Dawley rats were used in Phase 1 and four in Phase 2. Each was initially given isoflurane until unconscious. This was followed by preparation of the saphenous vein and the baseline basal BG was tested. The rats were then subcutaneously injected with 1 IU slow-acting insulin (Levemir), immediately following this they were placed on a heat-mat until they regained consciousness, before being housed in pairs in boxes. Blood collection (10 μl) was taken at 30-, 60-, 120-, 360-, and 465-minutes post-injection. BG testing was performed immediately at each time point using an Accu Chek BG monitor and strips. One rat didn't develop diabetes and one rat had kidney problems. In each case, the AWO (Animal Welfare Officer) was consulted and both rats were removed from the trial.

Delivery of Fast Acting Insulin (Actrapid) by Oral Capsule (Phase 1)

Four Sprague Dawley rats were used in this part of the trial. Each was initially given isoflurane until unconscious. This was followed by preparation of the saphenous vein and the first blood collection (10 μl) and BG testing. An enteric capsule (T=3) containing fast-acting insulin loaded in a cubic phase (25 μl cubic phase containing 1 IU insulin) was then introduced via oral gavage into the rat stomach. immediately following this they were placed on a heat-mat until they regained consciousness, before being housed in pairs in boxes. Blood collection (10 μl) was taken at 75-, 120-, 180-, 240-, 300- and 360- minutes, and BG testing was performed at each time point using an Accu Chek BG monitor and strips.

Delivery of Slow Acting Insulin (Levemir) by Oral Capsule (Phase 1)

Two Sprague Dawley rats were used in this part of the trial. Each was initially given isoflurane until unconscious. This was followed by preparation of the saphenous vein and the first blood collection (10 μl) and BG testing. An enteric capsule (T=3) containing slow-acting insulin loaded in a cubic phase (25 μl cubic phase containing 1 IU insulin) was then introduced via oral gavage into the rat stomach. Immediately following this they were placed on a heat-mat until they regained consciousness, before being housed in pairs in boxes. Blood collection (10 μl) was taken at 105-, 135-, 165-, 210-, 345- and 460- minutes, and BG testing was performed at each time point using an Accu Chek BG monitor and strips.

Delivery of Fast Acting Insulin (Actrapid) by Oral Capsule (Phase 2)

Four Sprague Dawley rats were used in this part of the trial. Each was initially given isoflurane until unconscious. This was followed by preparation of the saphenous vein and the first blood collection (10 μl) and BG testing. An enteric capsule (T=1) containing fast-acting insulin loaded in a cubic phase (25 μl cubic phase containing 1 IU insulin) was then introduced via oral gavage into the rat stomach. Immediately following this they were placed on a heat-mat until they regained consciousness, before being housed in pairs in boxes. Blood collection (10 μl) was taken at 45-, 75-, 120-, 160-, 220-, 260-, 300- and 360- minutes, and BG testing was performed at each time point using an Accu Chek BG monitor and strips.

Delivery of Slow Acting Insulin (Levemir) by Oral Capsule (Phase 2)

Two Sprague Dawley rats were used in this part of the trial. Each was initially given isoflurane until unconscious. This was followed by preparation of the saphenous vein and the first blood collection (10 μl) and BG testing. An enteric capsule (T=1) containing slow acting insulin loaded in a cubic phase (25 μl cubic phase containing 1 IU insulin) was then introduced via oral gavage into the rat stomach. Immediately following this they were placed on a heat-mat until they regained consciousness, before being housed in pairs in boxes. Blood collection (10 μl) was taken at 30-, 60-, 80-, 125-, 180-, 280-, 340-, 400- and 460- minutes, and BG testing was performed at each time point using an Accu Chek BG monitor and strips.

Results

Oral Delivery of Green Fluorescent Protein as a Model Drug

The proposed enteric capsule/cubic phase formulation for oral delivery of proteins was initially tested in a preliminary animal trial using a fluorescent protein. Measurement of fluorescence levels in the blood plasma allows for easy measurement of delivery of the drug to the bloodstream. Both green fluorescent protein (GFP) and red fluorescent protein (RFP) were identified as possible model drugs. To determine which was more suitable, the background blood plasma fluorescence in Sprague Dawley rats was determined for the GFP and RFP excitation and emission wavelengths, respectively. The mean blood plasma fluorescence for four rats was determined as 31106 RFU for the RFP excitation and emission wavelengths (570/620 nm), and 12123 RFU for the GFP excitation and emission wavelengths (488/509 nm). Due to the much higher background fluorescence for RFP, GFP was therefore used as a model protein drug for the initial animal trial.

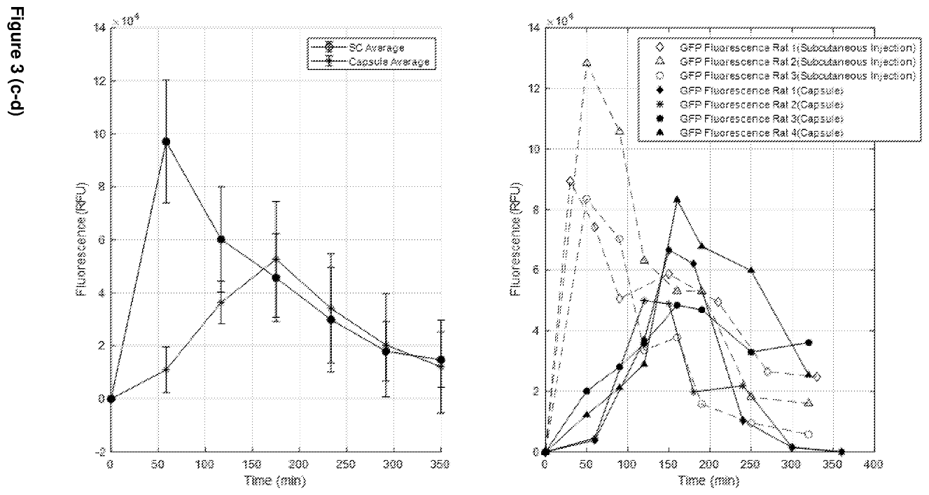

500 μL of 100 μg/ml GFP was administered via subcutaneous injection to three Sprague Dawley rats. 100 μL of blood was collected initially after 50-60 minutes, then every 30 minutes for 120 minutes, then every 60 minutes until the six-hour time point. It was not possible to have an initial time point earlier than 50-60 minutes due to the logistical constraints of the trial with all rats injected simultaneously. The plasma was separated from the collected blood samples, frozen, and fluorescence measured within 24 hours. Blood plasma fluorescence is plotted as a function of time following SC injection in FIG. 3A. An immediate increase in blood fluorescence is seen at the first time point at approximately 50-60 minutes; this then decays with time over the six hours of the trial. The variance between rats is relatively low and may be attributed to differences in blood chemistry between individual rats, for example, due to stress.

50 μg of GFP was then delivered to four rats encapsulated within a MO lipidic cubic phase (25 μl of 60 wt % lipid; 40% w/w water containing GFP at 2 mg/ml) and contained within an enteric capsule. Due to the size of the enteric capsule relative to the rat, the enteric capsule was delivered via oral gavage under anaesthetic. The water content of the lipidic cubic phase used (40 wt % water) was just under excess water conditions. A slightly reduced hydration level was chosen as excess water can cause early degradation of the enteric capsule. Blood collection was again taken periodically over a 6 hour period and the fluorescence measured, FIG. 3B. Similar to what was seen following SC injection the blood plasma fluorescence initially increased with time before decaying. The peak fluorescence measurement following oral delivery was delayed relative to that seen following SC injection, occurring between 120 minutes and 180 minutes, as expected for an oral administration route. The mean blood fluorescence following SC injection administration and capsule administration is provided in FIG. 3C, the error was calculated using the standard deviation across all rats in each experimental group. While the peak fluorescence measurement (50,000 RFU) was slightly lower, on average than that seen for SC injection (95,000), it was still consistent with effective delivery of the active protein via an oral administration route. The subsequent blood fluorescence decay rate was found to be similar to that following SC injection FIG. 3C, as expected. Variance in fluorescence between individual rats was noted but was not higher than that seen following SC injection.

The combined graph showing blood plasma fluorescence data from all rats following administration of GFP either via SC injection or loaded within an enteric capsule for oral delivery is provided in FIG. 3D. The total area under the curve was calculated as an indication of blood plasma levels of active GFP for each sample and is listed in Table 3.

For each oral delivery sample, this was expressed as a percentage of the average subcutaneous delivery, as shown in Table 3. The efficacy of oral administration relative to SC injection lay in the range 41.7% to 86.5% with an average efficacy of 62.1% for oral delivery, relative to delivery by SC injection.

Table 3 The total area under the curve for GFP fluorescence for all samples shown in FIG. 3D (subcutaneous and oral delivery). Average (%) bioavailability for oral delivery was calculated based on the average GFP fluorescence for subcutaneous injection delivery which was set to 100%.

| | Total GFP | |
|---|---|---|
| | Fluorescence | Percentage of |
| | (6 hours) | Subcutaneous (%) |
| Subcutaneous Injection 1 | 17663984 | N/A |
| Subcutaneous Injection 2 | 18166680 | N/A |
| Subcutaneous Injection 3 | 10248400 | N/A |
| Subcutaneous Injection Average | 15062032 | 100 |
| Capsule 1 | 7397610 | 49.1 |
| Capsule 2 | 6281865 | 41.7 |
| Capsule 3 | 10351880 | 68.7 |
| Capsule 4 | 13043553 | 86.5 |
| Capsule Average | 9367232 | 62.1 |

Oral Delivery of Actrapid (Fast Acting) Insulin

Based on the good bioavailability of GFP following oral administration a second animal trial was run using fast acting (Actrapid) insulin as a commercially relevant protein drug. 1 IU of fast acting (Actrapid) insulin was delivered to diabetic Sprague Dawley rats either via SC injection or oral (capsule) administration. Successful delivery of insulin may be determined directly by measuring the reduction in BG levels.

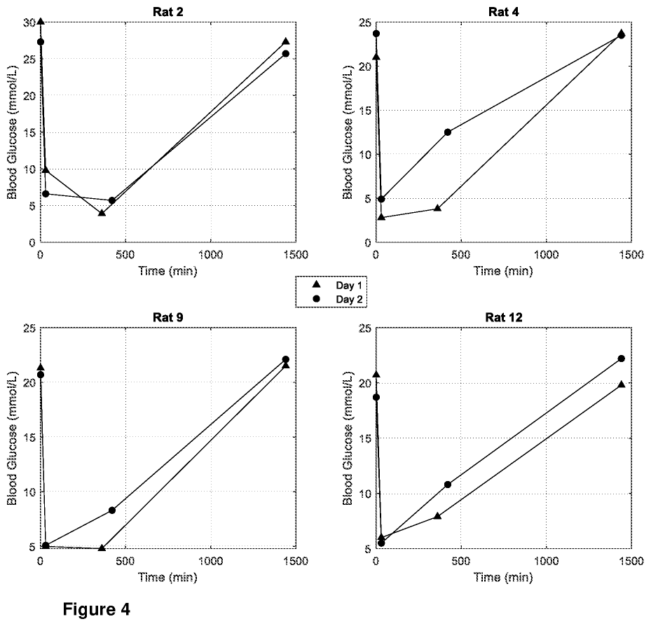

Diabetes was initially induced in the rats by an injection of 60 mg/kg STZ in a sodium citrate buffer followed by observation over 48 hours with testing of blood glucose first thing in the morning. Prior to induction of diabetes blood glucose levels for each rat were generally in the range 2 mmol/L-8 mmol/L. Once the blood glucose was found to be more than 14 mmol/L the rats were assumed diabetic. During the pre-trial period, blood glucose was then tested three times per day and insulin administered as required based on the blood glucose of each individual rat. Typical blood glucose readings during this period are provided in FIG. 4, for four randomly selected rats. All rats and data were approved by the Animal Welfare Officer to confirm that diabetes was successfully induced and that all rats were responding to insulin.

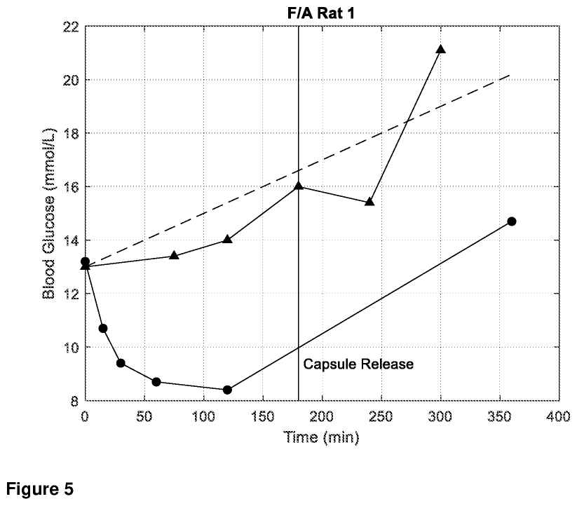

During the pre-trial period, the maximum blood glucose levels for diabetic rats were in the range 18-30 mmol/L, first thing in the morning due to their nocturnal nature. Following administration of a therapeutic dose of insulin by SC injection (15 IU) blood glucose levels dropped sharply over a period of 5 mins. BG levels remained below 14 mmol/L for approximately 450 minutes. After approximately 450 minutes, BG levels started to rise in a reasonably linear fashion indicating that most of the administered insulin had been purged from the system. Good reproducibility was observed in this increase in BG, between the four different rats and on two different days. The measured increase in BG (0.0167 mmol/L/min) was used to predict the rise in BG for rats in the absence of any treatment (shown as a dashed line in FIGS. 5-9).

To test the efficacy of insulin delivery via SC injection, 20 μl of Actrapid insulin at 50 IU/ml was initially administered to four diabetic rats. Following a recovery period of two days, the same four rats were then used for administration of Actrapid insulin contained within an enteric-coated capsule. 25 μl of lipidic cubic phase consisting of 60% wt MO, 40% wt Actrapid insulin at 100 IU/ml was used. A slightly reduced hydration level was chosen as excess water can cause early degradation of the enteric capsule. Insulin was encapsulated within this at 1 IU which is equivalent to 0.0384 mg. Insulin may be encapsulated within the lipidic cubic phase at this concentration with no impact on the underlying cubic phase architecture. Due to the small size and hydrophilic nature of the insulin, it should be contained within the aqueous channel region of the lipidic cubic phase.

Delivery of Fast Acting (Actrapid) Insulin to Diabetic Rats (Phase 1)

Comparative Experiment using a Perforated Capsule Shell using Rat 1

FIG. 5 For Rat 1, oral administration was conducted with a perforated enterically coated capsule shell. The result for this rat is consistent with the requirement for the combination of the enterically coated capsule shell and a nanocarrier comprising a lipidic cubic phase for successful delivery, as it is clear the cubic phase alone did not work following perforation of the capsule. The response of Rat 1 to the oral administration of insulin was not pronounced with blood glucose levels continuing to rise.

Experiment using Full Encapsulation using Rats 2-4

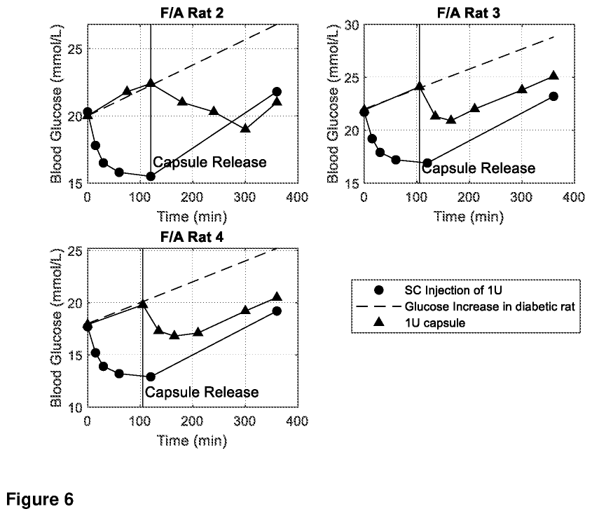

FIG. 6 plots BG levels for each of the three rats following administration of Actrapid insulin via SC injection (1 IU) or contained in a lipidic cubic phase within an enteric capsule (1 IU). As described in the section titled ‘Example 1—Materials and Methods’, for this trial each capsule was coated three times in the Enteric Coating mixture. The predicted rise in BG in the absence of any treatment (based on data in FIG. 4) is also plotted as a dashed line for all rats.

The initial BG level for Rats 2-4 varied between 18 mmol/l-22 mmol/L consistent with diabetic rats. Following administration of 1 IU of Actrapid insulin by SC injection an immediate decrease in BG levels was observed for all rats. BG levels continued to decrease over approximately 110 mins for all four rats before increasing again. The total decrease in BG varied between 4.2 mmol/L and 5.3 mmol/L with a final BG level in the range 8.9 mmol/L-17.2 mmol/L indicating successful administration of therapeutic levels of insulin. The rate of increase in BG after 360 mins is similar to the predicted rate of increase in the absence of insulin indicative that all administered Actrapid insulin has been purged from the animal. Reductions in BG observed are consistent with other animal trials of this kind.

For Rats 2-4, following oral (capsule) administration of Actrapid insulin BG levels initially continued to increase over a time period of approximately 100 to 110 mins. The rate of increase was essentially identical to that predicted in the absence of any treatment and is consistent with a longer time period for the insulin to reach the bloodstream following oral administration. After this time period, BG levels then started to decrease in a similar manner to what was observed with the SC injection over a time period of approximately 70-180 minutes. The overall decrease in BG was 3.6 mmol/L for Rat 2, 3.1 mmol/L for Rat 3 and 3.4 mmol/L for Rat 4, slightly smaller than what was observed for SC injection. After this time point levels of blood glucose began to increase, again at a similar rate to that predicted.

Delivery of Fast Acting (Actrapid) Insulin to Diabetic Rats Phase 2

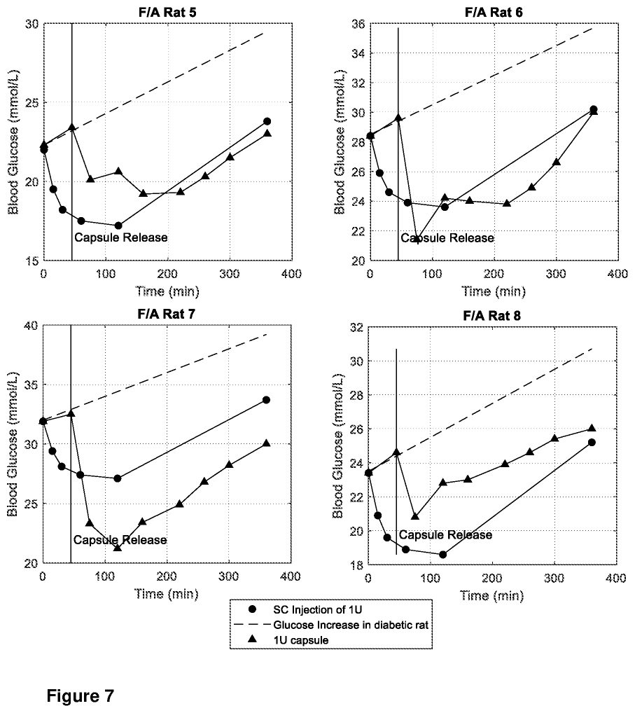

FIG. 7 For the second Phase using F/A insulin (Rats 5-8), the coating process for the enteric capsule was modified as described in the Methods section. Specifically, rather than the capsule being dipped in the enteric coating three times, it was only dipped once. We anticipate that this will result in a thinner coating which may break down earlier releasing insulin. In addition, removing two dipping steps may result in a more even coating between the different capsules. However, the thickness and uniformity of the coating was not explicitly measured.

Blood glucose data following SC injection of 1 IU insulin (FIG. 7) were very similar to those seen in Phase 1. Initial blood glucose levels were in the range of 22 mmol/l-33 mmol/L. Following administration of Actrapid insulin by SC injection an immediate decrease in BG levels was observed for all rats. BG levels continued to decrease over approximately 120 mins for all four rats before increasing again. The total decrease in BG varied between 4.1 mmol/L and 5.2 mmol/L with a minimum BG level in the range 17 mmol/L-26 mmol/. The rate of increase in BG after 120 mins is similar to the predicted rate of increase in the absence of insulin indicative that all administered Actrapid insulin has been purged from the animal. Reductions in BG observed are consistent with other animal trials of this kind.

For Rats 5-8, following oral (capsule) administration of Actrapid insulin BG levels initially continued to increase over a time period of approximately 60 mins. It is noted that this is a shorter lead time in comparison to Phase 1 (100-110 mins), consistent with the thinner enteric coating which should break down earlier. After this time period, BG levels then started to decrease in a similar manner to what was observed with the SC injection, and for oral administration in Phase 1. The overall decrease in BG was 6.2 mmol/l for Rat 5, 8.7 mmol/l for Rat 6, 11.4 mmol/l for Rat 7 and 4.6 mmol/l for Rat 8. This decrease is larger than what was observed in Phase 1 suggesting that the thinner enteric coating has led to improved uptake. After this time point levels of blood glucose began to increase, again at a similar rate to that predicted. All future experiments were therefore performed with capsules having this thinner enteric coating (FIG. 7).

Oral Delivery of Slow Acting (Levemir) Insulin

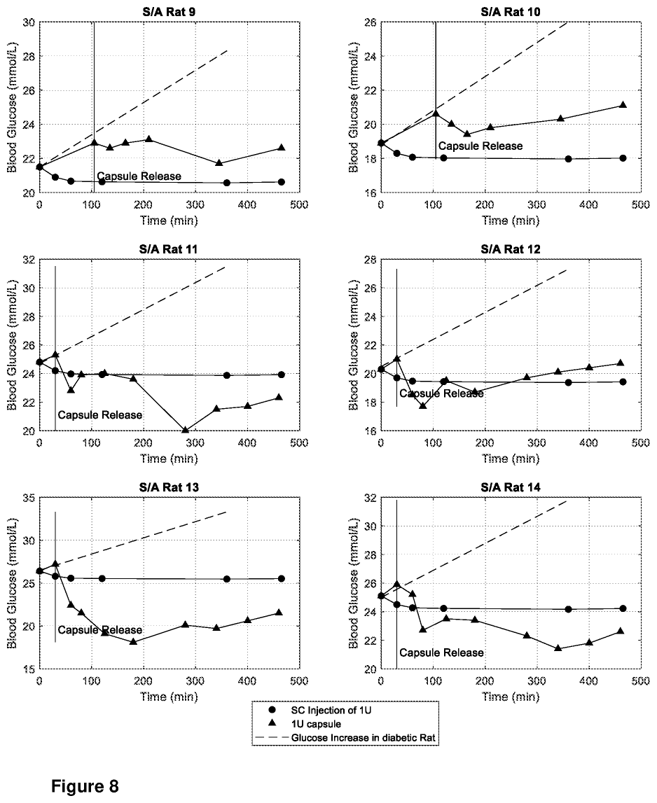

The ability of the lipidic cubic phase to deliver the slow acting insulin Levemir was also investigated. 20 μl of Levemir insulin at 50 IU/ml was initially administered to four rats via SC injection. Following a recovery period of two days, the same four rats were then used for administration of Levemir insulin encapsulated in cubic phase contained within an enteric-coated capsule. The lipidic cubic phase consisted of 60% wt MO, 40% wt Levemir insulin at a concentration of 100 IU/ml. Overall, the same dose of 1 IU of insulin was administered orally to each rat. FIG. 8 plots BG levels following administration of Levemir insulin via SC injection or contained in a lipidic cubic phase within an enteric capsule. Predicted subsequent increases in BG in the absence of treatment are plotted as a dashed line in each individual graph in FIG. 8. Note that results from Phase 1 and Phase 2 are provided together in FIG. 8. For Rats 9 and 10, results are from Phase 1 where the capsule was dipped three times in the enteric coating. For Rats 11-14, results are from Phase 2 where the capsule was dipped only once in the enteric coating.

Initial blood glucose levels were in the range of 19 mmol/l-25 mmol/L. Following administration of 1 IU Levemir insulin by SC injection an immediate decrease in BG levels was observed for all rats. BG levels continued to decrease over approximately 75 mins for all six rats before remaining steady over 480 min. The total decrease in BG varied between 1 mmol/L and 2 mmol/L with a minimum BG level in the range 18 mmol/L-25 mmol/L.

For Rats 9 and 10, following oral administration of Levemir insulin (T=3), the BG levels initially continued to increase over a time period of approximately 105 minutes before beginning to decrease. For Rats 11-14, (T=1) BG levels only increased over a 30-minute time period before a reduction in BG was observed. The rate of increase over this initial time period was essentially identical to that predicted in the absence of any treatment. Again, the difference in lead time before the insulin is released from the capsule and a decrease in BG is observed is consistent with the level of enteric coating. The lower lead time is associated with capsules having only one layer of the enteric coating as compared to three as seen in Phase 1 of the trial.

After this initial increase in BG, levels were then observed to decrease. We note that data are noisier than BG data following SC injection of either fast-acting or slow-acting insulin, or capsule administration of fast-acting insulin. Nevertheless, a clear decrease in BG levels are seen for all rats, and BG levels remain significantly lower than those predicted over 480 mins, the time period of the study. The overall decrease in BG was 1.4 mmol/l for Rat 1, 2.1 mmol/l for Rat 2, 6.1 mmol/l for Rat 3, 2.8 mmol/l for Rat 4, 8.2 mmol/l for Rat 5 and 4.3 mmol/l for Rat 6. For Rats 11, 13 and 14 where the enteric coating was thinner, at longer time points (>30-100 minutes) the decrease in BG is actually larger than that observed following SC injection.

We note that the slow-acting insulin can work for up to 14 hours. However, constraints of the animal trial (due to animal ethics restrictions on how long animals can be held under trial conditions) meant that BG was not analysed over these longer time periods. It is therefore impossible to compare the two administration methods over this time period.

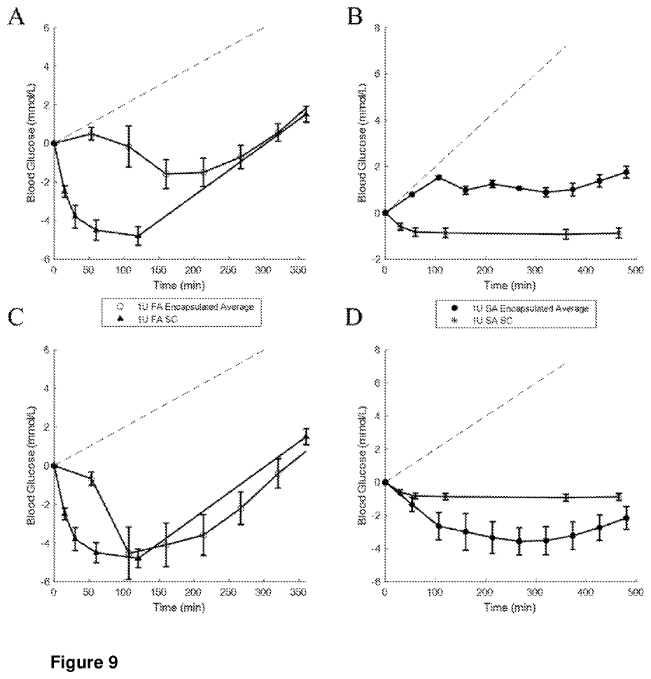

FIG. 9 plots the average blood glucose levels for A) fast-acting insulin—Rats 2-4 B) slow-acting insulin—Rats 9 and 10, C) fast-acting insulin—Rats 5-8, and D) slow-acting insulin—Rats 11-14. All data were normalized to set the initial BG reading to zero. For fast-acting, Rat 1 was removed from the average due to the capsule being perforated during induction. The area between each plotted graph and the predicted increase in blood glucose was calculated and is provided in Table 3. This was used to determine the total reduction in blood glucose (as a measure of efficacy) for both SC injection and oral delivery compared to a standard increase in blood glucose in a diabetic rat.

Table 4 Total BG decrease calculated from the area between the graph and the predicted blood glucose average increase (dashed line) for the fast-acting insulin SC injection and the fast-acting insulin encapsulated. Total BG decrease calculated from the area between the graph and the predicted blood glucose average increase (dashed line) for the slow-acting insulin SC injection and the slow-acting insulin encapsulated.

| | Total decrease in BG | Percentage |
|---|---|---|
| | (360 mins) relative to | of SC |
| | predicted increase | injection |
| Insulin Administration Route | (mmol/L) | (%) |
| Fast-Acting Insulin (SC injection) | 2062.1 | 100 |
| Fast Acting Insulin (capsule - T = 3) | 1156.8 | 56.1 |
| Fast Acting Insulin (capsule - T = 1) | 1920.2 | 93.1 |
| Slow Acting Insulin (SC injection) | 1518.9 | 100 |
| Slow Acting Insulin (capsule - T = 3) | 789.5 | 52.0 |
| Slow Acting Insulin (capsule - T = 1) | 2398.9 | 157.9 |

In summary, efficacious oral delivery of model therapeutic agent was achieved using a combination of an enterically coated formulation of a lipid nanocarrier in the cubic phase. Experiments using GFP initially demonstrated that the enteric capsule/lipidic cubic phase combination allowed for successful oral delivery of model therapeutic agents, with an efficacy of approximately 62% when comparing oral delivery to SC injection of GFP. This trial also allowed the testing of the coating level of the enterically coated capsule, testing a slightly thicker enteric coating achieved by dipping the capsules twice. However, efficacy of delivery can only be measured indirectly using fluorescence.

Subsequent animal studies using insulin then demonstrated the success of the enterically coated capsule/lipid nanocarrier in the cubic phase as a combination to deliver insulin and derivatives thereof. Successful delivery was demonstrated for both fast-acting and slow-acting forms of insulin. Delivery was tested using two different levels of coating—a thicker coating achieved by dipping the capsule three times and a thinner coating achieved by dipping the capsule once. For the thicker coating, the efficacy of oral delivery of fast-acting insulin was 56% relative to SC injection, and 52% for slow acting insulin. The thinner coating was associated with both a faster time for the drug to reach the bloodstream and a higher efficacy, 93% for fast-acting insulin rising to 158% for slow-acting insulin. It was not possible to test the slow-acting insulin over the full time period of 16 hours and this efficacy value may therefore be slightly higher than that achieved over the full time period.

### Example 3 Preparation of the Dosage Form with Small Molecule and Protein Therapeutic Agents using Lipids in the Cubic Phase

Materials.

Monoolein (MO) (97%, Sigma), Eudragit L 100 (Evonik), Capsules S9 (Torpac), Meropenem (Sigma), vancomycin (Sigma), Human Coagulation Factor 10 (Resolving Images), Human Growth Hormone (MyBioSource). Male Sprague Dawley rats were sourced from the ARC animal facility (Perth, Australia). HGH, HCFX, vancomycin and meropenem Elisa Kits were sourced from BMA.

Capsule Preparation 1—(Eudragit L 100)

For the preparation of the lipidic cubic phase with therapeutic agent encapsulated, a known amount of monoolein (typically 50 mg) was added to a 100 μl ml syringe. A solution of protein/hormone in PBS (pH adjusted to 4 using HCL) was added to another 100 μl ml syringe in the ratio 38:62 w/w drug solution:lipid. The contents of both syringes were mixed using a specialized syringe mixing attachment.

A diluent mixture was created using Acetone (268 ml), Isopropanol (400 ml) and Milli Q water (43 ml) and poured into a 3 L beaker. A high torque mixer was used to mix Eudragit L 100 (62.5 g) into the diluent and was slowly mixed for 60 min. Triethyl citrate (6.25 g) was added and stirred for a further hour before being passed through a 0.5 mm sieve to yield the enteric coating mixture. Capsules were coated in this mixture once using a dipping tray. After the enteric coating was dried each capsule part was put in the capsule holder, the enteric mixture was pipetted into and out of the individual capsule parts. Capsules were filled with 25 μl of the drug-loaded LCP as described above resulting in a final amount of 1 U per capsule. The coating thickness on the inside and outside was calculated to be approximately 160 μm by solving the change in mass for the geometry of a capsule by radius=((surface_area/(2*pi*r))−length)/2.

Capsule Preparation 2—(Eudragit L 100-55)

For the preparation of the lipidic cubic phase with the therapeutic agent encapsulated, a known amount of monoolein (typically 50 mg) was added to a 100 μl ml syringe. A solution of therapeutic agent in PBS (pH adjusted to 5 using HCL) was added to another 100 μl ml syringe in the ratio 38:62 w/w drug solution:lipid. The contents of both syringes were mixed using a specialized syringe mixing attachment.

A diluent mixture was created using Acetone (268 ml), Isopropanol (400 ml) and Milli Q water (43 ml) and poured into a 3 L beaker. A high torque mixer was used to mix Eudragit L 100-55 (62.5 g) into the diluent and was slowly mixed for 60 min. Triethyl citrate (6.25 g) was added and stirred for a further hour before being passed through a 0.5 mm sieve to yield the enteric coating mixture. Capsules were coated in this mixture once using a dipping tray. After the enteric coating was dried each capsule part was put in the capsule holder, the enteric mixture was pipetted into and out of the individual capsule parts. Capsules were filled with 25 μl of the drug-loaded LCP as described above. The coating thickness on the inside and outside was calculated to be approximately 160 μm by solving the change in mass for the geometry of a capsule by radius=((surface_area/(2*pi*r))−length)/2.

Delivery of Protein Hormone Therapeutic Agents

Delivery of Human Coagulation Factor X by Subcutaneous Injection

Four male Sprague Dawley rats were used as controls. Each rat was anaesthetised with isoflurane followed by preparation of the saphenous vein. The rats were given 0.5 mg/kg of human coagulation factor X via SC injection, immediately following this they were placed on a heat-mat until they regained consciousness, before being housed in pairs in boxes. Blood collection (150 μl) was taken via the saphenous vein at 0, 30-, 70-, 120-,180-, 240- and 360-mins post-injection. The blood concentration of the protein in the blood was tested using the relevant ELISA kit for human coagulation factor X.

Delivery of Human Coagulation Factor X by Oral Capsule

Eight male Sprague Dawley rats were used. Each rat was anaesthetised with isoflurane followed by preparation of the saphenous vein and the first blood collection (150 μl). An enteric capsule with a thick polymer coating of approximately 160 μm containing 0.5 mg/kg of the human coagulation factor X encapsulated in MO lipid cubic phase was then introduced via oral gavage into the rat stomach. Immediately following this they were placed on a heat-mat until they regained consciousness, before being housed in pairs. Blood collection (150 μl) was taken via the saphenous vein at 0, 35-, 50-, 120-,180-, 240- and 360-mins post-injection

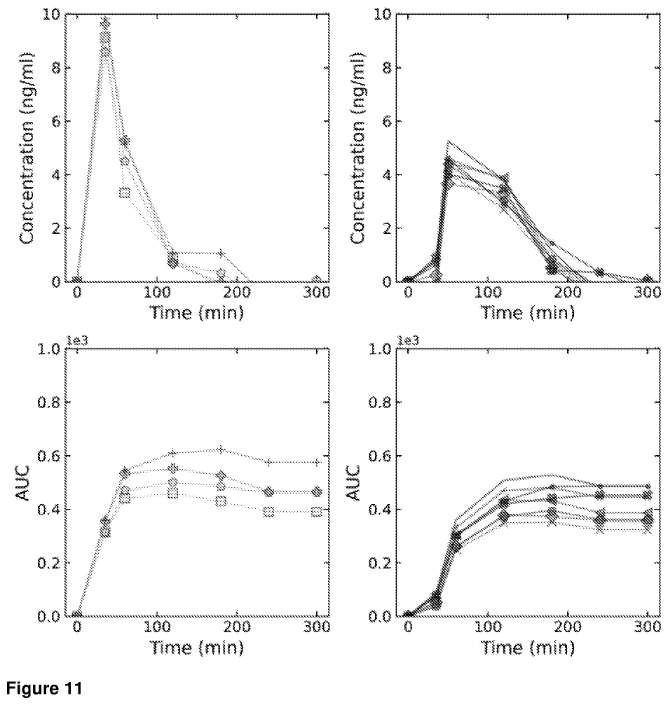

As shown in FIG. 11, for all four rats, the drug concentration either does not increase or increases by only a small amount, over the first 35 mins of the trial. The drug concentration subsequently rises sharply to a maximum value in the range of 3.7-5.2 ng/ml at 50 mins. This is followed by a decrease to 0 ng/ml over the subsequent 4 hours, with baseline drug concentrations reached by 300 min. The maximum drug plasma concentration achieved was 47% of that following SC injection. The overall bioavailability was 88.52% of that following SC injection.

Delivery of Human Growth Hormone by Subcutaneous Injection

Four male Sprague Dawley rats were used as controls. Each rat was anaesthetised with isoflurane followed by preparation of the saphenous vein. The rats were given 1 mg/kg of human growth hormone via SC injection, immediately following this they were placed on a heat-mat until they regained consciousness, before being housed in pairs in boxes. Blood collection (150 μl) was taken via the saphenous vein at 0, 30-, 70-, 120-, 180-, 240- and 360-mins post-injection. The blood concentration of the protein in the blood was tested using the relevant ELISA kit for human growth hormone.

Delivery of Human Growth Hormone by Oral Capsule

Eight male Sprague Dawley rats were used. Each rat was anaesthetised with isoflurane followed by preparation of the saphenous vein and the first blood collection (150 μl). An enteric capsule with a thick polymer coating of approximately 160 μm containing 1 mg/kg of the human growth hormone encapsulated in MO lipid cubic phase was then introduced via oral gavage into the rat stomach. Immediately following this they were placed on a heat-mat until they regained consciousness, before being housed in pairs. Blood collection (150 μl) was taken via the saphenous vein at 0, 35-, 50-, 120-,180-, 240- and 360-mins post-injection

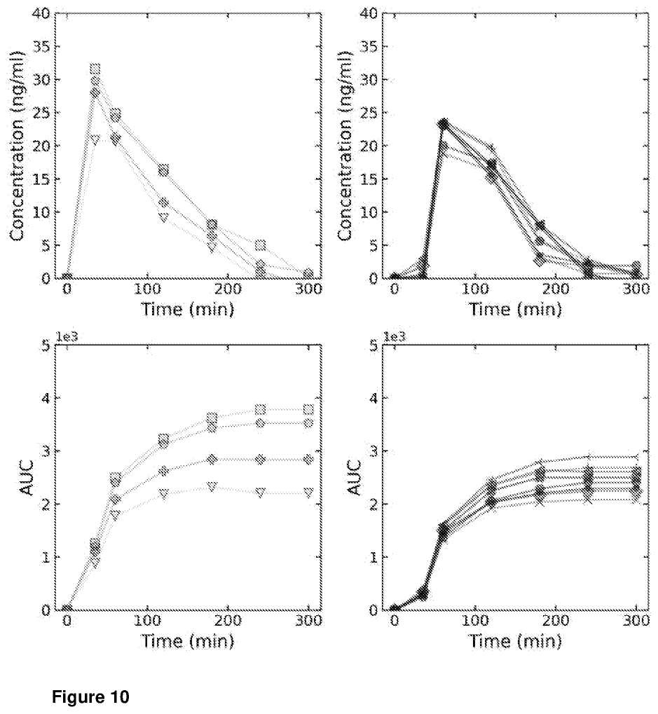

As shown in FIG. 10, for all four rats the drug concentration either does not increase or increases by only a small amount, over the first 35 mins of the trial. The drug concentration subsequently rises sharply to a maximum value in the range of 19-24 ng/ml at 50 mins. This is followed by a decrease to 0 ng/ml over the subsequent 3 hours, with baseline drug concentrations reached by 240 min. FIG. 10(C) plots the area under the drug response curve (AUC) for the data provided in (A). (D) plots the area under the drug response curve (AUC) for the data provided in (B). It is representative of the total delivered protein/hormone which is integral to determine overall biodistribution as a factor of time. Following SC injection, the total amount of protein delivered increases with time, reaching a plateau at approximately 180 min. Following administration via oral capsule, a short lag period of 30 mins is observed before the total protein delivered starts to increase also reaching a plateau around 240 mins. There was also less variability in the results amongst the different rats following oral delivery. The maximum drug plasma concentration achieved was 81% of that following SC injection. The overall bioavailability was 87.2% of that following SC injection.

Delivery of Small Molecule Antibiotic Therapeutic Agents

Delivery of Meropenem by Subcutaneous Injection

Four male Sprague Dawley rats were used as controls. Each rat was anaesthetised with isoflurane followed by preparation of the saphenous vein. The rats were given 1 mg/kg of meropenem via SC injection, immediately following this they were placed on a heat-mat until they regained consciousness, before being housed in pairs in boxes. Blood collection (150 μl) was taken via the saphenous vein at 0, 30-, 70-, 120-, 180-, 240- and 360-mins post-injection. The blood concentration of the protein in the blood was tested using the relevant ELISA kit for meropenem.

Delivery of Meropenem by Oral Capsule

Eight male Sprague Dawley rats were used. Each rat was anaesthetised with isoflurane followed by preparation of the saphenous vein and the first blood collection (150 μl). An enteric capsule with a thick polymer coating of approximately 160 μm containing 1 mg/kg of meropenem encapsulated in MO lipid cubic phase was then introduced via oral gavage into the rat stomach. Immediately following this they were placed on a heat-mat until they regained consciousness, before being housed in pairs. Blood collection (150 μl) was taken via the saphenous vein at 0, 35-, 50-, 120-, 180-, 240- and 360-mins post-injection.

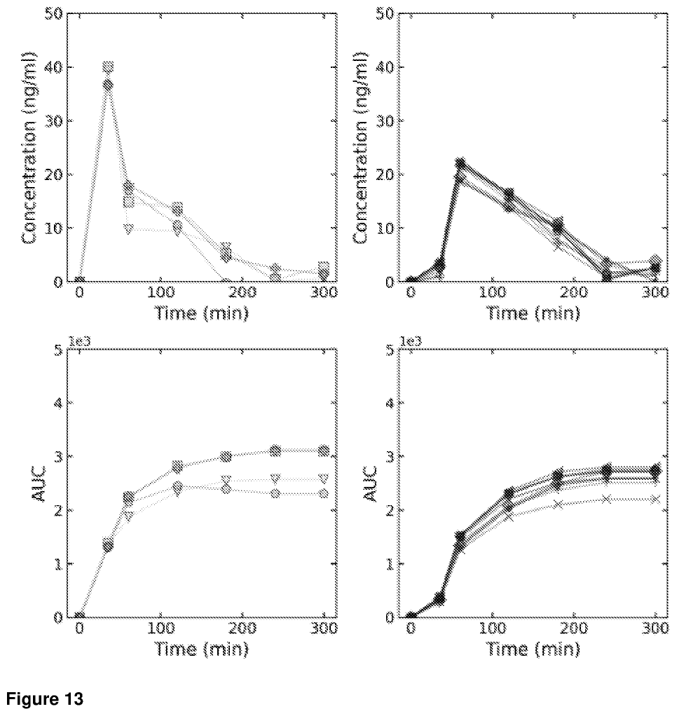

As shown in FIG. 13, for all four rats the drug concentration either does not increase, or increases by only a small amount, over the first 35 mins of the trial. The drug concentration subsequently rises sharply to a maximum value in the range 19-23 ng/ml at 50 mins. This is followed by a decrease to 0 ng/ml over the subsequent 2 hours, with baseline drug concentrations reached by 180 min. The maximum drug plasma concentration achieved was 55% of that following SC injection. The overall bioavailability was 58% of that following SC injection.

Delivery of Vancomycin by Subcutaneous Injection

Four male Sprague Dawley rats were used as controls. Each rat was anaesthetised with isoflurane followed by preparation of the saphenous vein. The rats were given 15 mg/kg of vancomycin via SC injection, immediately following this they were placed on a heat-mat until they regained consciousness, before being housed in pairs in boxes. Blood collection (150 μl) was taken via the saphenous vein at 0, 30-, 70-, 120-, 180-, 240- and 360-mins post-injection. The blood concentration of the protein in the blood was tested using the relevant ELISA kit for each of the proteins.

Delivery of Vancomycin by Oral Capsule

Eight male Sprague Dawley rats were used. Each rat was anaesthetised with isoflurane followed by preparation of the saphenous vein and the first blood collection (150 μl). An enteric capsule with a thick polymer coating of approximately 160 μm containing 15 mg/kg of vancomycin encapsulated in MO lipid cubic phase was then introduced via oral gavage into the rat stomach. Immediately following this they were placed on a heat-mat until they regained consciousness, before being housed in pairs. Blood collection (150 μl) was taken via the saphenous vein at 0, 35-, 50-, 120-, 180-, 240- and 360-mins post-injection.

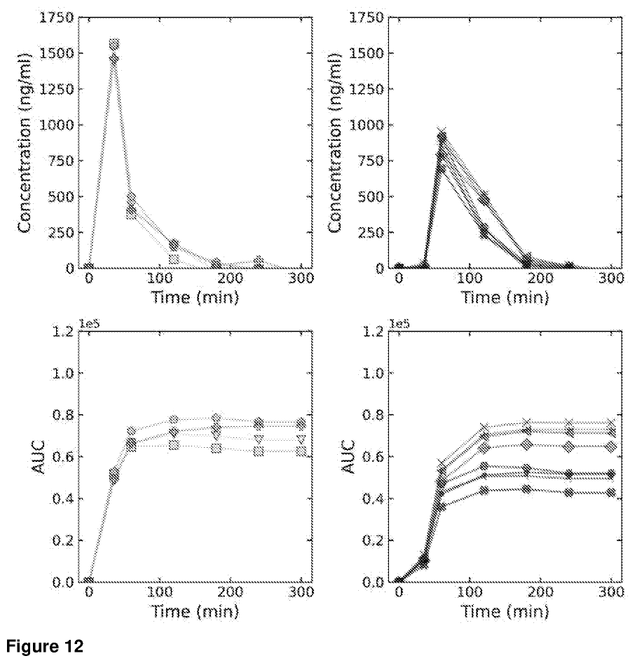

As shown in FIG. 12, for all four rats, the drug concentration either does not increase or increases by only a small amount, over the first 35 mins of the trial. The drug concentration subsequently rises sharply to a maximum value in the range 790-960 ng/ml at 50 mins. This is followed by a decrease to 0 ng/ml over the subsequent 2 hours, with baseline drug concentrations reached by 180 min. The maximum drug plasma concentration achieved was 57% of that following SC injection. The overall bioavailability was 73.93% of that following SC injection.

### Example 4 Structural Analysis of Therapeutic Agents with Lipids in the Form of a Mesophase

Small-Angle X-Ray Scattering (SAXS)

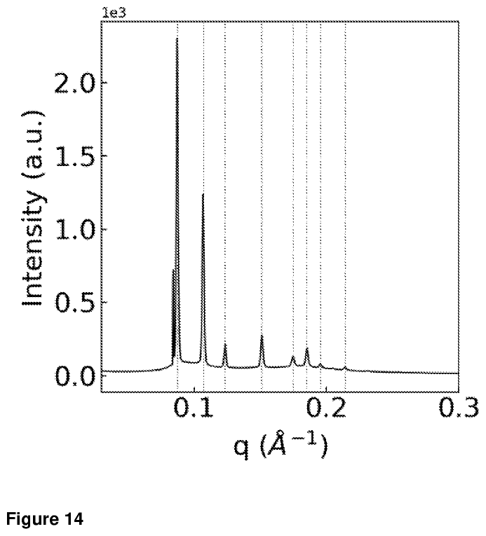

Synchrotron SAXS experiments were performed at the Australian synchrotron to determine the structure of the lipid mesophase used within each capsule. Excess cubic phase from animal trials was taken to the SAXS/WAXS beamline and data are provided in FIGS. 14-16. With 1 mg/ml concentrations for HGH, HCFX and 15 mg/ml for vancomycin.

FIG. 14 is a 1D SAXS pattern for human growth hormone (1 mg/ml) encapsulated in MO at an aqueous phase content of 38% the √2, √3, √4, √6, √8, √9, √10, √11 Bragg peaks of a QIID cubic phase (crystallographic space group Pn3m) are indicated by dashed lines. The lattice parameter of the QIID phase is calculated to be 101.8 Å.

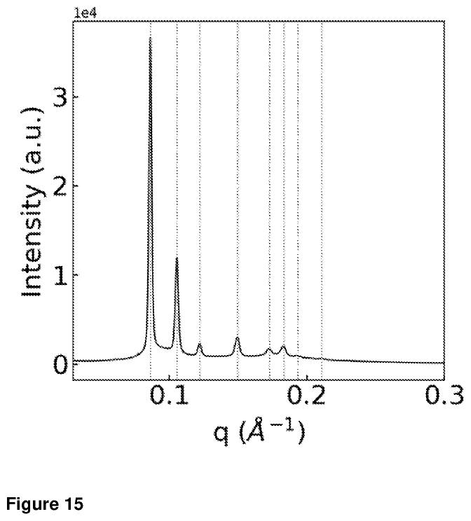

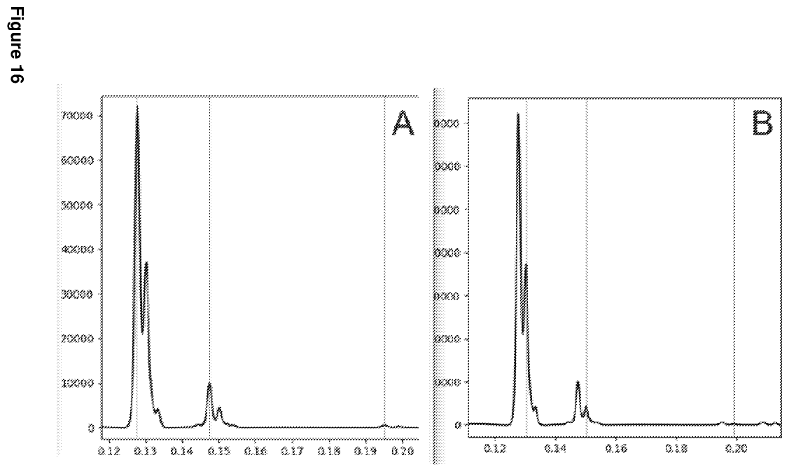

FIG. 15 is a 1D SAXS patterns for human coagulation factor X (1 mg/ml) encapsulated in MO at an aqueous phase content of 38%. The √2, √3, √4, √6, √8, √9, √10, √11 Bragg peaks of a QIID cubic phase (crystallographic space group Pn3m) are indicated by the dashed lines. The lattice parameter of the QIID phase is calculated to be 103.0 Å.

FIG. 16 is a 1D SAXS patterns for vancomycin (15 mg/ml) encapsulated in MO at an aqueous phase content of 38%. Two coexisting QIIG phases (crystallographic space group Ia3d) are observed—Bragg peaks in the ratio √6, √8, √14 are indicated in (A) (lattice parameter=120.5 Å) and (B) (lattice parameter=118.2 Å).

Plot/Data/Experiment Conventions

All data are analysed on a Clariostar Plate Reader to determine concentration against a standard curve for the relevant drug.

Concentrations provided in FIGS. 10-13 are the measured concentration of the drug in the blood plasma (determined using specific Elisa kits) and are in units of ng/ml. The initial drug concentration measured at the 0 time point has been set to zero in each case and other data scaled relative to this.

### Example 5 Studies of Insulin and Lipid Nanocarriers in the Form of the Cubic Phase

Monoolein (oleoyl-rac-glycerol) (MO) (>99% purity), phytantriol (PT) (>99% purity), Pluronic F127 (>99%), human recombinant insulin (>99%), octadecyl rhodamine B chloride (>99%), chymotrypsin (>99%), fluorescein isothiocyanate (FITC) labelled insulin (>99%), and Phosphate buffered saline (PBS) (>99%) were purchased from Sigma Aldrich (Australia).

Formulation of Cubic Phase

An aqueous solution of insulin in phosphate buffered saline (PBS) (various concentrations between 0.5 mg/ml and 50 mg/ml) was mixed with molten phytantriol or molten monoolein to form the bulk cubic phase with insulin. This was achieved by filling a 0.5 mL syringe with the insulin solution (50 wt % for MO and 30 wt % for PT samples) and an 0.5 mL syringe with the molten lipid (50 wt % for MO and 70 wt % for PT) and mixing by passing through a syringe coupler as described. The resulting mixture was visually observed to be cloudy due to the presence of excess water, consistent with the known excess water points of both lipids (48% for MO and 28% for PT). To ensure both cubic phases were formulated at just under excess water conditions a small amount of lipid was subsequently added and mixed using the syringe mixer until the formulated cubic phase was visually observed to be clear with no evidence of any cloudiness. Based on the known phase diagrams of MO and PT the final cubic phase has a water content of approximately 48% for MO and 28% for PT.

Small Angle X-Ray Scattering (SAXS)

200 nL aliquots of this cubic phase were dispensed onto the surface of a Corning 96 well plate using a Mosquito LCP machine equipped with a humidity chamber (TTPLabtech, UK). The plate was sealed with a 200 lm plastic seal (Molecular Dimensions). SAXS data were obtained at the SAXS/WAXS beamline at the Australian Synchrotron using a 96-well plate set-up custom designed within our laboratory. The x-ray beam had a wavelength of k=1.12713 Å (10.000±0.002 keV) with dimensions of 250 μm×120 μm and flux of 5×10¹² photons s⁻¹. The sample detector used was a Pilatus 1-M at a distance of 900 mm from the sample. The temperature was maintained at 25° C. using a Peltier-controlled system. Automatic data acquisition for a single plate takes around 10 mins. The AXCESS software package, developed at Imperial College London was used for SAXS data analysis. 2D diffraction images were integrated to produce 1D plots of intensity vs scattering vector q; calibration was performed using silver behenate, d=58.38 Å as a standard. For the QII D phase (crystallographic space group Pn3m) adopted by both MO and PT at all insulin concentrations studied, the p (110), (111), (200) and (211) Bragg peaks in the ratio √2, √3, √4, √6, were used to calculate the lattice parameter based on known positions of the peaks. The lattice parameter is determined from the peak position q using the following equation:

$$\\ a = \frac{2 {π ( {h}^{2} + {k}^{2} + {l}^{2} )}^{0.5}}{q}$$

The error is calculated based on SAXS measurements on three (3) different samples.

Circular Dichroism (CD)

SR-CD measurements were conducted in the 170-350 nm region using the CD beamline of the synchrotron radiation source ASTRID2 at ISA, Centre for Storage Ring Facilities at Aarhus University (Denmark). Suprasil Quartz cells (Hellma GmbH & Co., Germany) of nominal 0.1 mm path length (actual calibrated path lengths=102.3 and 97.7 mm), requiring an approximately 30 μl sample volume, were used for measurements at 37° C. in a temperature-regulated sample holder. For enzyme degradation experiments run in the far UV region 20 μl chymotrypsin solution at 0.02 mg/ml was injected into the sample holder to surround the 10 μl of bulk cubic phase with insulin encapsulated at 0.2 mg/ml. For enzyme degradation experiments run in the near UV region 20 μl chymotrypsin solution at 0.05 mg/ml was injected into the sample holder to surround the 10 μl of bulk cubic phase with insulin encapsulated at 0.5 mg/ml. The baseline measurements (cubic phase in absence of insulin) were obtained at 1 nm increments in wavelength, 2.15 sec averaging time and 6 scans. The sample measurements were obtained at 1 nm increments in wavelength, 1 sec averaging time and 1 scan for each sample. Data were mildly smoothed with a 5-point moving mean in Matlab. The online service DichroWeb was used for secondary structure content analysis of the CD spectra using the CONTIN routine and the SP175 reference set.

Fluorescence Recovery after Photobleaching (FRAP)

FRAP was performed on a Nikon confocal microscope. For measurements on the MO and PT pure cubic phase, octadecyl rhodamine B chloride (100 mg/ml) was mixed with MO and PT in the ratio lipid:aqueous phase 50:50 for MO and for PT. A bleaching and background region of interest (ROI) was used to selectively photobleach an area of the cubic phase and the fluorescence recovery curve was recorded with time. The excitation/emission wavelengths used were 553/627 nm for the octadecyl rhodamine B chloride. For measurements on insulin within the cubic phase, the MO or PT cubic phase was loaded with FITC labelled insulin (5 mg/ml). A bleaching and background region of interest (ROI) was used to selectively photobleach an area of the cubic phase and the fluorescence recovery curve was recorded with time. The excitation/emission wavelengths used were 490/525 nm, respectively, for the FITC labelled insulin. The open source Matlab code frap_analysis_2p5 was used to determine diffusion coefficients μ from FRAP data.

Release from Bulk Cubic Phase

An aqueous solution of insulin in PBS (5 mg/ml) was mixed with molten phytantriol or molten monoolein to form the bulk cubic phase (100 mg) with encapsulated insulin as described in the section: Formulation of cubic phase. This mixture was then injected into a 1.5 mL Eppendorf tube and centrifuged at 1200 RPM for 1 min to achieve a flat surface. The height of this surface was measured to determine the diameter for subsequent surface area calculations. 150 μl of water was added on top of the cubic phase and immediately removed for T=0 time point. 150 μl of supernatant fluid was removed every 30 min over a 12 hour period for analysis and replaced with a further 150 ml of water. A final reading was taken at 22 hour. The concentration of insulin in the supernatant fluid was calculated at each time point using the absorbance at 280 nm (A280 analysis) on a Nanodrop 2000 (Thermo Fisher).

Structural Studies of Insulin Lipid in the Lipid Nanocarrier in the Form of a Cubic Phase

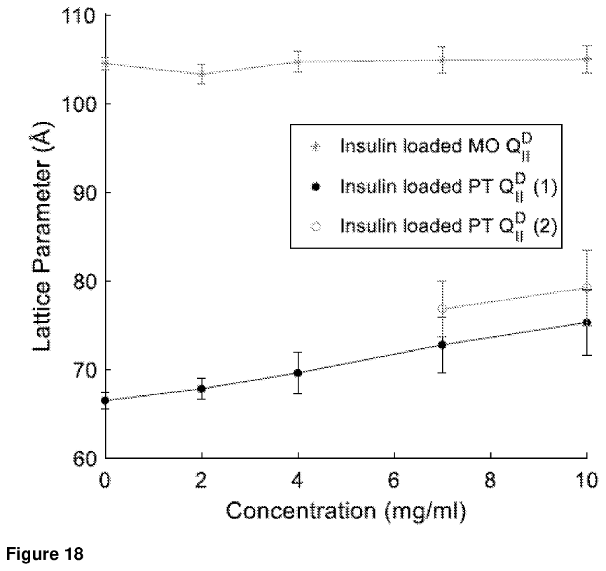

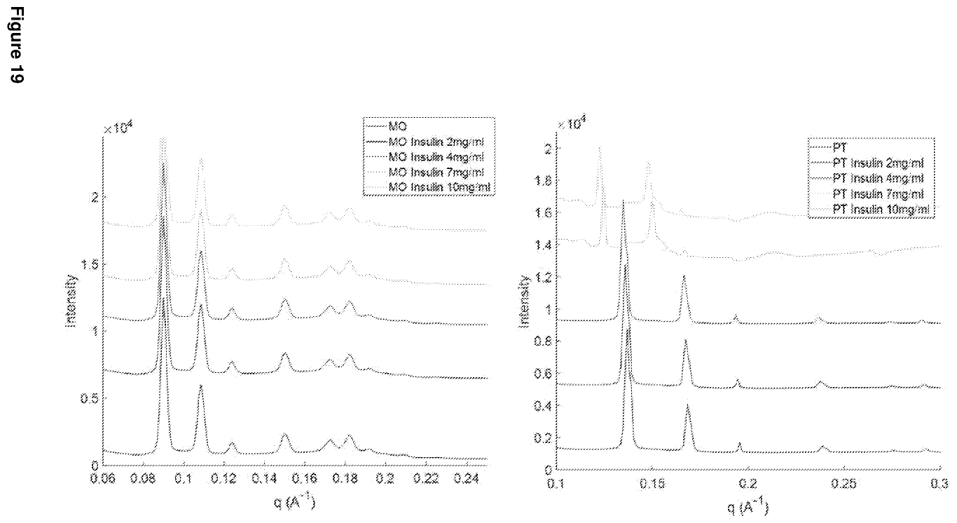

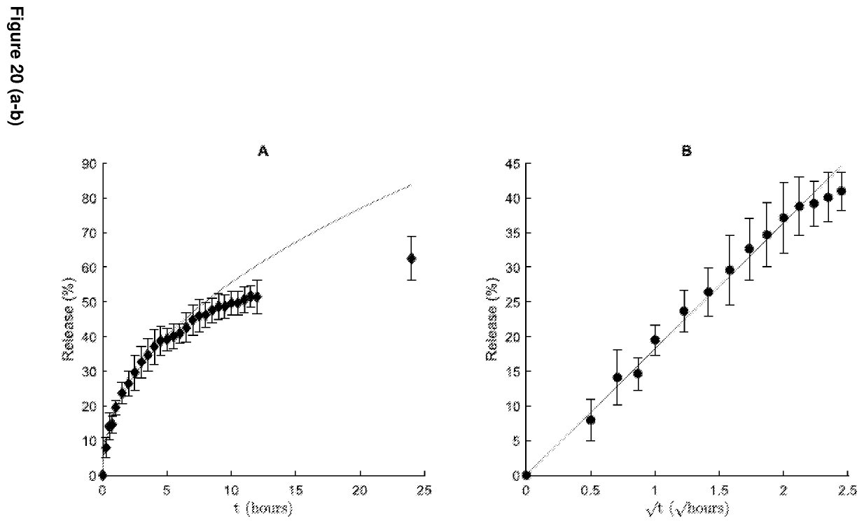

Synchrotron SAXS was used to characterise the structure of the lipidic cubic phase formed by MO and PT following encapsulation of insulin at a range of concentrations up to 10 mg/ml. The phase adopted, and associated lattice parameter at 25° C. are shown in FIG. 18 Samples were run in triplicate. Representative diffraction patterns for the MO and PT cubic phase with encapsulated insulin are provided in FIG. 19. The bulk cubic phase formed by MO was a Q_{II}D phase with a lattice parameter of 104 Å, in agreement with the literature. Addition of insulin did not significantly affect the underlying nanostructure at any concentration studied, up to 10 mg/ml insulin. The lattice parameter remained reasonably constant with increasing insulin concentration FIG. 18 while Bragg peaks remain well defined and do not broaden over the concentration of insulin used (FIG. 19) PT also adopted a Quo phase in excess water, with a smaller lattice parameter of 66 Å. Insulin within the PT nanocarrier in the form of the cubic phase resulted in an increase in lattice parameter which was proportional to the insulin concentration. By 10 mg/ml the lattice parameter had increased to 75 Å, an increase of 14%. Error bars are observed to increase with increasing insulin concentration, as is common for proteins which have a disruptive effect on the cubic phase. For PT, a second Q_{IID} cubic phase was also observed at higher protein concentrations >7 mg/ml. Based on the average intensity of the √2 and √3. The second Q_{II D} Dcubic phase is the minor phase. The lattice parameter of the second Q_{II D} cubic phase was slightly higher than the original phase and may represent the inhomogeneous distribution of the protein.

The increased disruptive effect of insulin on the PT cubic nanostructure may reflect an increased geometrical mismatch between the insulin and the aqueous channel within which it is located. In aqueous solution, insulin is known to exist mainly in the dimeric form with a measured hydrodynamic diameter of 36 Å. The dimer would, therefore, be expected to fit easily within the aqueous channels of the MO cubic phase (51 Å), consistent with retention of the original cubic lattice parameter for this system at all insulin concentrations studied. In contrast, accommodation of the insulin dimer within the smaller 24 Å channels of the PT cubic phase would result in considerable strain in the cubic lattice which could be relieved by a swelling of the system and the observed increase in lattice parameter with increasing protein concentration.

Diffusion of Insulin in the Lipidic Nanocarrier in the Form of the Cubic Phase

The diffusion coefficient of the protein within the lipid matrix, depends on both the size of the water channels and the presence of “bottlenecks” in the cubic phase, is known to be directly related to observed drug release rates. The diffusion coefficient of FITC-labelled insulin was therefore measured using FRAP, both in solution and when encapsulated within the lipidic cubic phase, Table 5.

TABLE 5
The diffusion coefficient of octadecyl rhodamine B chloride was
also measured within the MO and PT cubic phase using FRAP.

| | Diffusion | |
|---|---|---|
| | Coefficient × | |
| Sample | 10⁻⁶ cm² s⁻¹ | R² |
| Octadecyl rhodamine B | 0.97 | 0.99 |
| chloride in MO (Q_{II}^{D} phase) | | |
| Octadecyl rhodamine B | 0.59 | 1 |
| chloride in PT (Q_{II}^{D} phase) | | |
| Insulin (solution) | 6.56 | 0.99 |
| Insulin (MO Q_{II}^{D} phase) | 2.29 | 0.99 |
| Insulin (MO Q_{II}^{D} phase interface) | 2.89 | 0.99 |
| Insulin (PT Q_{II}^{D} phase) | 4.17 | 0.99 |
| Insulin (PT Q_{II}^{D} phase interface) | 4.36 | 0.98 |

The diffusion coefficient of octadecyl rhodamine B chloride in each cubic phase, listed in Table 5, strongly correlated with the corresponding lipid self-diffusion coefficient. In this respect, we note that the measured diffusion coefficients for the octadecyl rhodamine B chloride in MO (0.97×10⁻⁶ cm² s⁻¹) and PT (0.59×10⁻⁶ cm² s⁻¹) were highly similar to lipid self-diffusion coefficients for MO and PT measured using NMR in a previous study (1.2×10⁻⁶ cm² s⁻¹ and 0.3×10⁻⁶ cm² s⁻¹ respectively). Diffusion coefficients for lipids and insulin within the bulk lipid cubic phase and at the water interface as determined by FRAP. The diffusion coefficient of free insulin, as measured using FRAP, (6.6×10⁻⁶ cm² s⁻¹) is in reasonable agreement with that previously provided in the literature in water (1.5×10⁻⁶ cm² s⁻¹) and the diffusion coefficient as determined from the Stokes-Einstein equation for an insulin dimer (1.4×10⁻⁶ cm² s⁻¹).

Measured diffusion coefficients within the cubic matrix were only slightly lower than those measured in water. This may reflect the fact that the continuous aqueous channel network in the cubic phase allows for facile diffusion of the insulin in three dimensions, particularly given that the insulin dimer is not significantly larger than the water channel radius. We note that the diffusion coefficient for insulin within the bulk PT cubic phase (4.17×10⁻⁶ cm² s⁻¹) was higher than that for MO (2.29×10⁻⁶ cm² s⁻¹). This is a slightly counter-intuitive result, as the smaller aqueous channel size for PT would be expected to hinder diffusion. Previous studies on the diffusion of glucose at 37° C. have shown that diffusion coefficient of glucose was higher in the cubic phase formed by MO (1.9×10⁻⁶ cm² s⁻¹) than in PT (7.3×10⁻⁷ cm² s⁻¹). However, we note reasonable variation in previously published values with a diffusion coefficient of 3.6×10⁻⁷ cm² s⁻¹ also reported for diffusion of glucose in MO, although this sample was formulated at 30% w/w aqueous phase which is far below the excess water point for MO (48%). Partial location of the insulin in such “nanopockets” within the PT matrix could result in the slightly increased diffusion coefficient for insulin in PT (and the increased release rate described in the following section). Diffusion coefficients were measured both at the centre of the bulk cubic phase and at the water interface. For both lipids, the diffusion coefficient was approximately 15% higher at the interface compared to in the bulk. This may be attributed to the large gradient in insulin concentration between the bulk phase and the surrounding water

Release of Insulin from the Lipid Nanocarrier in the Bulk Cubic Phase

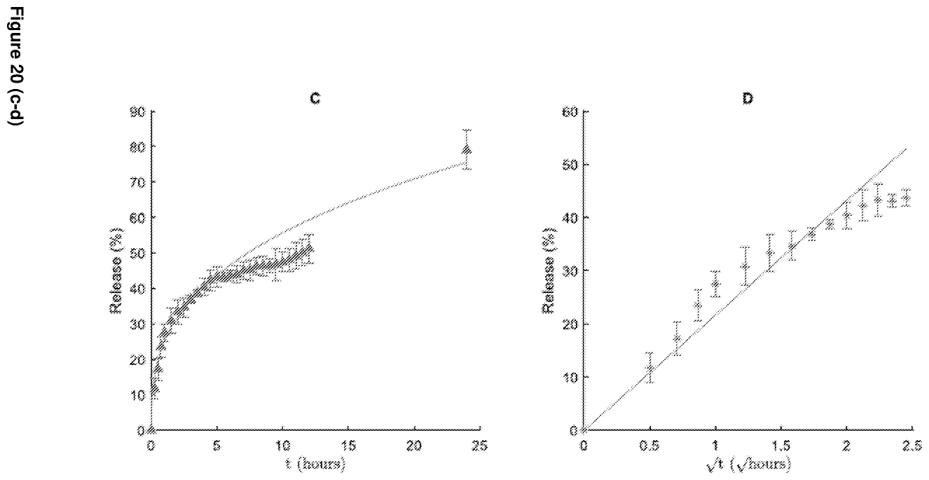

The encapsulation efficiency of insulin in the cubic phase was measured as 92.6% and 83.9% for MO and PT, respectively. The lipid nanocarrier mesophase with insulin was then exposed to an aqueous sink and the concentration of the released insulin measured over a 24 h period. The percentage of insulin released with time is provided in FIG. 20A for release from MO and in FIG. 20C for release from PT. For both the MO and PT cubic phases, insulin release was initially fast and the rate gradually decreased with time, as shown in FIG. 20A and 20C, respectively. The release of insulin from PT was generally faster, particularly in the early stages with 20% release achieved in ˜2 h from PT vs ˜4 h for MO. This is consistent with the faster diffusion coefficient measured for insulin in PT. Over longer timescales, approximately 50% release was achieved from MO over a 24 h period versus 60% release from PT. To investigate the release mechanism from the mesophase, the release profiles were fit against the Ritger-Peppas model:

$$\\ \frac{{M}_{t}}{{M}_{0}} = k \times {t}^{n}$$

over the first 7 h (17 time points). Mₜ, M₀, k and n are the released quantity at time t, initial quantity encapsulated, fitting coefficient and fitting exponent, respectively. This allows for the coupling of multiple release mechanisms (e.g., diffusion-controlled, zero-order) where the diffusional exponent n, is indicative of the release mechanism and geometry of the interface and the fitting coefficient k reflects characteristics of both the matrix and the solute. The determined fitting parameters are shown in Table 6. The calculated exponents for MO and PT were 0.45 and 0.41, respectively, consistent with diffusion controlled or Fickian release (diffusional exponent less than 0.5).

TABLE 6
Determined fitting parameters from insulin release studies.
n is the fitting exponent and k is the fitting coefficient
used in the Ritger-Peppas model. D is the determined
diffusion co-efficient from the Higuchi model.

| Sample | n | k | D (×10⁻⁶ cm² s⁻¹) |
|---|---|---|---|
| MO | 0.45 ± 0.31 | 0.88 ± 0.06 | 4.61 ± 0.26 |
| PT | 0.41 ± 0.43 | 1.33 ± 0.15 | 6.85 ± 0.34 |

Diffusion controlled release from a thin film or slab can similarly be described by the Higuchi Equation which is defined by:

$$\\ Q = 2 \times {C}_{0} \times \sqrt{\frac{D \times t}{π}}$$

where Q, C₀ and D are the released concentration per unit area, initial concentration per unit volume and the diffusion coefficient, respectively. Diffusion control dictates that the release profile against √t should be linear. As shown in FIG. 20B and D, the release shows a reasonable correlation to a linear fit, further indicating that the release was predominantly diffusion controlled. The diffusion coefficient for insulin in MO and PT was determined using the Higuchi Equation (Drelease) and is shown in Table 6. The determined parameters were compared to those found during FRAP analysis (DFRAP). The DFRAP and Drelease values were 2.89×10⁻⁶ cm²/s and 4.61×10⁻⁶ cm²/s for release of insulin from MO. For PT the DFRAP and Drelease values were 4.36×10⁻⁶ cm²/s and 6.85×10⁻⁶ cm²/s, respectively.

The lipidic Nanocarrier in the Form of the Cubic Phase Protects Encapsulated Insulin Against Enzymatic Degradation.

The ability of the cubic matrix to protect encapsulated insulin against enzymatic degradation was investigated. Chymotrypsin, which is typically found in the small intestine, was utilized as a model enzyme. Synchrotron CD was used to determine the evolution of the secondary insulin structure following exposure to chymotrypsin both in solution, and when within either a MO or PT based cubic phase.

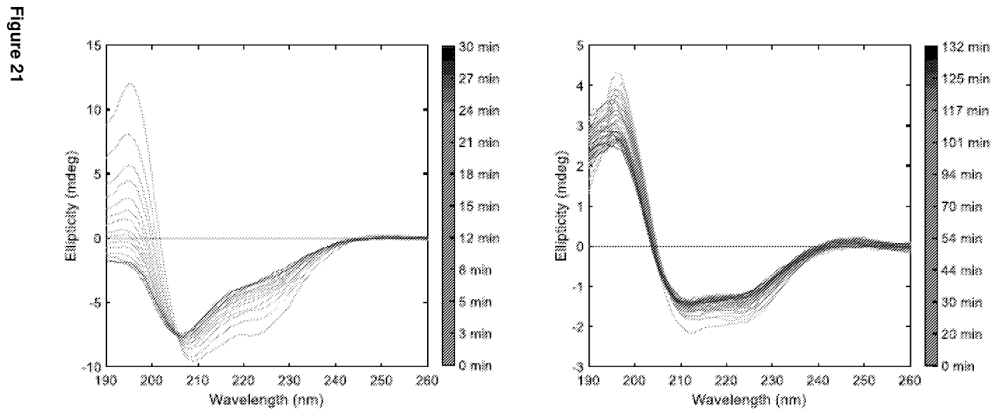

For insulin in solution, FIG. 21 (left), the initial spectrum consists of a large maximum at ˜200 nm and a double minimum at 208 and 222 nm, consistent with a structure that is largely alpha-helical. Dichroweb analysis, Table 6, yielded a structure that was 89% alpha-helical. Following the addition of chymotrypsin, immediate changes to the secondary structure were observed. The maximum near 200 nm was observed to decrease in a steplike fashion over the 30 min timescale, while the double minimum is gradually replaced by a single minimum at lower wavelengths, consistent with destruction of the alpha-helices. 12 min after addition of the enzyme, the percentage of alpha-helices had dropped to 41% and the unordered structures had increased to 41%, Tables 7 and 8.

Tables 7 and 8 immediately below are Dichroweb analysis of synchrotron CD spectra for insulin in water (0.2 mg/ml) at various time points following the addition of chymotrypsin (0.02 mg/ml).

| Time (Min) | α-helix(%) | β-sheet(%) |
|---|---|---|
| 0 | 89 | 0 |
| 2 | 68 | 0 |
| 6 | 51 | 2 |
| 12 | 41.2 | 3.3 |

| Time (Min) | β-turns(%) | Unordered(%) | NRMSD |
|---|---|---|---|
| 0 | 3 | 8 | 0.024 |
| 2 | 9 | 22 | 0.118 |
| 6 | 13 | 33 | 0.166 |
| 12 | 14 | 40.5 | 0.328 |

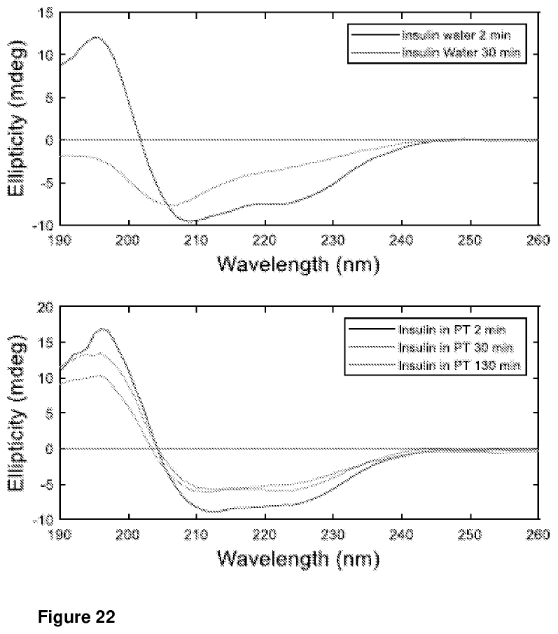

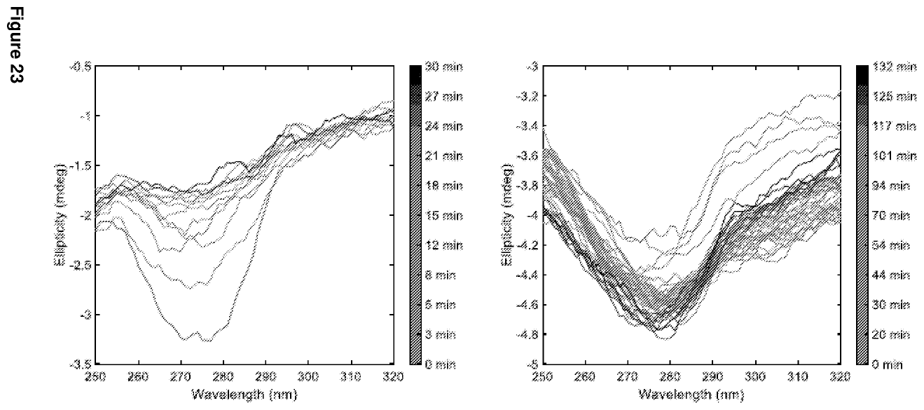

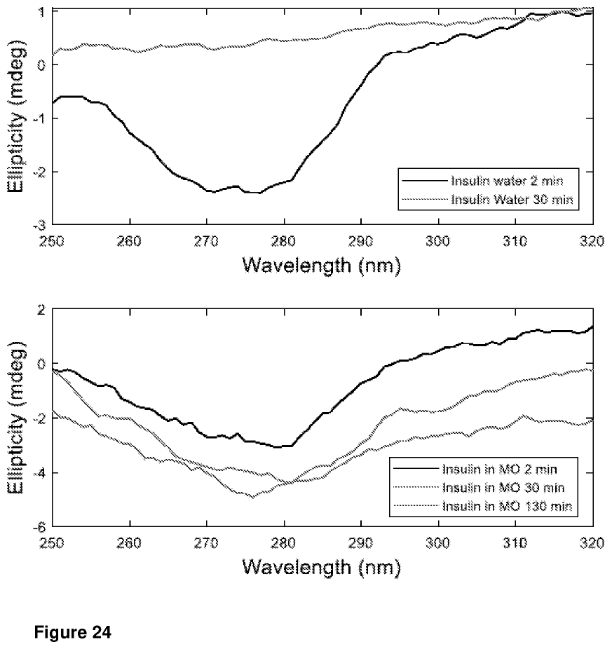

Dichroweb analysis was not possible at longer timescales due to extensive degradation of the protein structure. 30 min after addition of the chymotrypsin the CD spectrum consists of a single minimum at approximately 208 nm consistent with a structure that is largely disordered, FIG. 21. Changes to the secondary structure of insulin encapsulated in phytantriol following the addition of chymotrypsin are shown in figure over a 132 min timescale. Selected spectra for insulin in PT at 2, 30 and 130 min are provided in FIG. 22 to aid visualisation of differences between the two samples. While some reduction in the intensity of the maximum near 200 nm and the double minimum was observed, we note that changes to the secondary structure of insulin are significantly reduced when encapsulated in phytantriol. Although dichroweb analysis was not reliable for these spectra due to the presence of the phytantriol, 30 min after addition of chymotrypsin the spectrum for insulin in PT is still largely a-helical, while that for insulin in water is largely unordered, FIGS. 21 and 22. Even 2 hours after addition of the chymotrypsin, the spectrum for insulin encapsulated in PT still shows a maximum near 200 nm and a double minimum consistent with the retention of a-helical structures (FIGS. 21 and 22) demonstrating the ability of the lipidic cubic phase to protect the insulin against enzymatic attack over a physiologically relevant timescale. A similar analysis was not possible for monoolein as the cubic phase formed by MO is more opaque than the PT cubic phase resulting in significant noise in the data at lower wavelengths. However, a near-UV spectrum, related to the tertiary structure of the protein, was obtained for insulin in solution and in MO (FIG. 23). Selected spectra for insulin in solution and in MO at various time points are provided in FIG. 24 to aid visualisation of differences between the two samples. In solution, the tertiary structure of insulin undergoes immediate change following the addition of chymotrypsin with a reduction in the minimum at 270 nm, again consistent with the destruction of the a-helical secondary structure. This minimum has essentially disappeared by 9 min after addition of the enzyme. While a similar reduction in the intensity of the minimum is observed for insulin in monoolein, the changes observed are more gradual and over a longer timescale, again indicating the protective nature of the lipid matrix. The minimum at 270 nm may still be observed even 132 min after addition of the chymotrypsin, FIG. 24.

Degradation of the insulin by the chymotrypsin could occur via two different mechanisms. Insulin could diffuse out of the bulk cubic phase and interact with the chymotrypsin in the surrounding fluid. Alternatively, the chymotrypsin could diffuse into the bulk cubic phase and through the aqueous channel network. We note that the protection occurred over the first two hours. As shown by the black line in FIG. 20 A and C, only approximately 15% and approximately 25% of the insulin was released from MO and PT, respectively, over this time period. Therefore, the diffusion rate of the enzyme through the bulk cubic phase, which depends on the enzyme size, charge and hydrophobicity, is the dominant factor restricting enzymatic degradation.

The cubic mesophase nanocarrier offered significant protection to the loaded protein for a period of up to 2 hours. We note that some insulin may be released from the cubic phase during this time period, potentially resulting in fast solution-based enzymatic degradation for the released protein. However, release data obtained in this study indicate that only approximately 20% of the insulin should be released over this 2-hour time period, with the majority of the loaded insulin retained within the protective lipid nanocarrier. This exemplifies the potential of the lipidic cubic phase to protect water-soluble proteins, small molecules and peptide-based drugs of a range of molecular weights against the enzymatically destructive environment of the human gastrointestinal tract.

1. A dosage form for oral delivery of a therapeutic agent to a subject, the dosage form comprising:
(i) a lipid nanocarrier formulation, the lipid nanocarrier formulation comprising the therapeutic agent, and wherein the lipid nanocarrier formulation is in the form of a mesophase; and
(ii) an enteric coating encapsulating the lipid nanocarrier formulation.

2. The dosage form of claim 1, wherein the mesophase comprises a reverse bicontinuous cubic phase, a primitive cubic phase, double diamond cubic phase, a gyroid cubic phase, a hexagonal phase, a reverse hexagonal phase, cubosomes or hexosomes.

3. The dosage form of claim 1, wherein the lipid nanocarrier comprises a lipid selected from the group consisting of a mono-, di-, or tri-substituted glycerol, a charged lipid, a long chain lipid, a branched lipid and a glycolipid.

4. The dosage form of claim 3, wherein the charged lipid is dioleoyl-3-trimethylammonium propane (DOTAP) present in an amount of up to and including 10% of the lipid nanocarrier formulation.

5. The dosage form of claim 1, wherein the lipid nanocarrier comprises lipids of the following formula I: 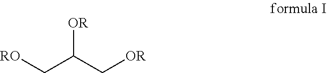
wherein at least one R is formula II, and the reaming R groups are independently selected from a hydrogen or formula II: 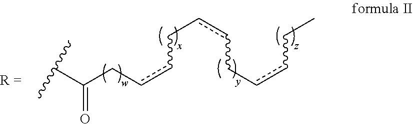
wherein w, x, y and z are independently selected from the group consisting of 0, 1, 2, 3, 4, 5, 6, 7, 8, 9, 10, 11 and 12; wherein a broken line represents the presence or absence of a bond; and wherein a wavy bond indicates E or Z bond geometry in the presence of a bond.

6. The dosage form of claim 1, wherein the lipid nanocarrier comprises lipids of the following formula III 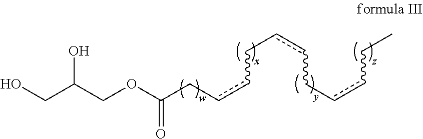
wherein w, x, y and z are independently selected from the group consisting of 0, 1, 2, 3, 4, 5, 6, 7, 8, 9, 10, 11 and 12; wherein a broken line represents the presence or absence of a bond; and wherein a wavy bond indicates E or Z bond geometry in the presence of a bond.

7. The dosage form of claim 1, wherein the lipid nanocarrier comprises lipids selected from the following group: monoolein, phytantriol and monopalmitolein.

8. The dosage form of claim 7, wherein monoolein is present in about 40% to 80% weight of the formulation.

9. The dosage form of claim 7, wherein phytantriol is present in about 60% to 75% weight of the formulation.

10. The dosage form of claim 1, wherein the therapeutic agent is selected from the list consisting of insulin or derivative thereof, a steroidal hormone, antimicrobial such as an antibiotic, a protein such as a hormone, and a peptide such as a neuropeptide and wherein the insulin or derivative thereof is selected from the group consisting of glargine (Lantus, Basaglar, Toujeo), detemir(Levemir), degludec(Tresiba), NPH(Humulin N, Novolin N, Novolin ReliOn Insulin N), rapid-acting insulin and short-acting insulin.

11. (canceled)

12. (canceled)

13. The dosage form of claim 1, wherein the nanocarrier comprises aqueous channels of sizes 1 nm to 17 nm.

14. (canceled)

15. The dosage form of claim 14, wherein the enteric coating is soluble at range of about pH 5.0 to pH 6.0.

16. (canceled)

17. The dosage form of claim 1, wherein the lipid nanocarrier formulation is aqueous.

18. The dosage form of claim 13, wherein the lipid nanocarrier formulation has a water content in a range of 1% to 70% weight of the lipid nanocarrier formulation.

19. The dosage form of claim 1, wherein the lipid nanocarrier formulation has a water content in a range of up to and including 48% weight of the lipid nanocarrier formulation and wherein the lipid is monoolein, or has a water content in a range of up to and including 48% weight of the lipid nanocarrier formulation and wherein the lipid is phytantriol.

20. The dosage form of claim 1, wherein the dosage form is a capsule comprising a filling comprising the lipid nanocarrier formulation and a shell encapsulating the filling, the shell coated with the enteric coating.

21. (canceled)

22. (canceled)

23. (canceled)

24. (canceled)

25. A method for preparing the dosage form of claim 1, comprising the steps of:
a) providing the lipid nanocarrier formulation; and
b) encapsulating the lipid nanocarrier formulation in an enteric coating.

26. The method of claim 17 wherein the lipid nanocarrier formulation is prepared by contacting by contacting the lipid, the therapeutic agent and an aqueous solvent under conditions sufficient to promote formation of a lipid mesophase.

27. The method of claim 18, wherein the lipid to aqueous solvent ratio is about 60:40 w/w.

28. (canceled)

29. (canceled)

30. (canceled)

31. A method for treating or preventing diabetes mellitus, a condition mediated by bacterial infection, a condition mediated by human growth hormone, or a condition mediated by blood coagulation which comprises administering to a subject in need the dosage form of claim 1, and wherein the therapeutic agent is present in a therapeutically or prophylactically effective amount.

32. (canceled)
